(19) United States
(12) Patent Application Publication
(10) Pub. No.: US 2006/0257420 A1
(43) Pub. Date: Nov. 16, 2006

(54) Methods of preparation and composition of peptide constructs useful for treatment of autoimmune and transplant related host versus graft conditions

(75) Inventor: Daniel H. Zimmerman, Bethesda, MD (US)

Correspondence Address: HAHN & VOIGHT PLLC, 1012 14TH STREET, NW, SUITE 620, WASHINGTON, DC, 20005, US

(73) Assignee: CEL-SCI Corporation, Vienna, VA (US)

(21) Appl. No.: 11/443,314

(22) Filed: May 31, 2006

Related U.S. Application Data
(63) Continuation-in-part of application No. 11/298,718, filed on Dec. 12, 2005.
Application No. 11/298,718 is a continuation of application No. 10/111,645, filed on Apr. 26, 2002, now Pat. No. 6,995,237.

(51) Int. Cl. A61K 39/00; C07K 14/47

(52) U.S. Cl. 424/185.1; 530/324

(57) ABSTRACT

The invention is related to peptide constructs, i.e., polypeptides obtained by linking together two or more peptides based on or derived from different molecules, which are useful in the treatment or prevention of autoimmune diseases, asthma, allergies, and host versus graft (or graft versus host) rejection, as well as to compositions containing same, methods for producing same and methods for using same; wherein the peptide constructs have the formula
P₁-x-P₂ where
P₁ is a peptide associated with autoimmune disease, allergy, asthma, host-versus-graft rejection, myocarditis, diabetes, and immune-mediated disease, which binds to an antigen receptor on a set or subset of T cells;
P₂ is a peptide which will cause a Tₕ2 directed immune response by the set or subset of T cells to which the peptide P₁ is attached or which will bind to a T cell receptor which will cause the set or subset of T cells to which the peptide P₁ is attached to initiate, but not complete, an immune response causing the set or subset of T cells to undergo anergy and apoptosis; and
   x is a direct bond or linker for covalently bonding P₁ and P₂.

This invention is a continuation-in-part application of U.S. patent application Ser. No. 11/298,718 filed on Dec. 12, 2005, which is a continuation of U.S. patent application Ser. No. 10/111,645 having a § 371 date of Apr. 26, 2002, now U.S. Pat. No. 6,995,237, the content of which is incorporated herein by reference. Two compact discs required under Rule § 1.821(c) and (e) are made part of the specification. The first compact disc is the “Sequence Listing” and is an exact duplicate of the second compact disc, which is the Computer Readable Copy (CRF) required under Rule § 1.821(e). The subject matter of the “Sequence Listing” is incorporated herein by reference

### INTRODUCTION

The invention is related to peptide constructs, i.e., polypeptides obtained by linking together two or more peptides based on or derived from different molecules, which are useful in the treatment or prevention of autoimmune diseases, asthma, allergies, and host versus graft (or graft versus host) rejection, as well as to compositions containing same, methods for producing same and methods for using same.

### BACKGROUND

Autoimmune conditions are characterized by the body attacking itself by mounting an immune response against itself. The goal of these treatments is to turn off, dampen or redirect this inappropriate immune response. The goal of a vaccination approach is to eliminate, reduce or redirect an immune response against self-antigens. As noted by a recent Institutes of Medicine report, autoimmune diseases are a major target area proposed for vaccines and are the third major area for expenditure of healthcare in the US behind cancer and cardiovascular diseases.

Various antigens often with defined epitopes recognized for some Human Leukocyte Antigens (HLA) genotypes, have been identified, including those associated with Insulin Dependent Diabetes Mellitis (IDDM), Rheumatoid Arthritis (RA) (e.g. collagen type II 390-402 IAGFKGEQGPKGE (SEQ ID NO.1) wherein amino acids are provided in PRT/1 code shown in Table D-1 of Appendix D of User's Manual for PatentIn 3.3 (Version 2.4), Computer Sciences Corporation (Jul. 30, 2004), Systemic Lupus Erythematousis (SLE), Ankyosing Spondylitis (AS), Pemphigus Vulgaris (PV) (epidermal cell adhesion molecule desmoglein 190-204), Multiple Sclerosis (MS), Myelinproteolipid (MPL) (peptide sequence KNIVTPRT (SEQ ID NO.2), certain types of psoriasis, and uveoretinitis (Hammer et al., HLA class I peptide binding specificity and autoimmunity. 1997, Adv. Immunol, 66:67 Tisch et al., Induction of Glutamic Acid Decarboxylase 65-Specific Th2 Cells and Suppression of Autoimmune Diabetes at Late Stages of Disease Is Epitope Dependent 1999, J. Immunol. 163:1178; Yoon et al., Control of Autoimmune Diabetes in NOD Mice by GAD Expression or Suppression in β Cells 1999, Science 284:1183; Ruiz et al., Suppressive Immunization with DNA Encoding a Self-Peptide Prevents Autoimmune Disease: Modulation of T Cell Costimulation 1999, J. Immunol., 162:3336; Krco et al., Identification of T Cell Determinants on Human Type II Collagen Recognized by HLA-DQ8 and HLA-DQ6Transgenic Mice 1999, J. Immunol., 163:1661). In other cases, peptides are known that induce in animals, a condition similar to ones found in humans, such as GDKVSFFCKNKEKKC (SEQ ID NO.3) for antiphospholipid antibodies associated with thrombosis (Gharavi et al., GDKV-Induced Antiphospholipid Antibodies Enhance Thrombosis and Activate Endothelial Cells In Vivo and In Vitro 1999, J. Immunol., 163:2922) or myelin peptides for experimental autoimmune encephalitis (EAE) as a model for MS (Ruiz et al., supra, Araga et al., A Complementary Peptide Vaccine That Induces T Cell Anergy and Prevents Experimental Allergic Neuritis in Lewis Rats 1999, J. Immunol., 163:476-482; Karin et al., Short Peptide-Based Tolerogens Without Self-Antigenic or Pathogenic Activity Reverse Autoimmune Disease 1999, J. Immunol., 160:5188; Howard et al., Mechanisms of immunotherapeutic intervention by anti-CD40L (CD154) antibody in an animal model of multiple sclerosis 1999, J. Clin. Invest., 103:281).

Moreover, glutamic acid decarboxylase (GAD) and specific peptides have been identified associated with IDDM (Tisch et al., supra; Yoon et al., supra). Many of these conditions are also characterized by elevated levels of one or more different cytokines and other effectors such as Tumor Necrosis Factor (TNF) (Kleinau et al., Importance of CD23 for Collagen-Induced Arthritis: Delayed Onset and Reduced Severity in CD23-Deficient Mice 1999, J. Immunol. 162:4266; Preckel et al., Partial agonism and independent modulation of T cell receptor and CD8 in hapten-specific cytotoxic T cells 1998, Eur. J. Immunol., 28:3706; Wooley et al., Influence of a recombinant human soluble tumor necrosis factor receptor FC fusion protein on type II collagen-induced arthritis in mice 1993, J. Immunol., 151:6602) as well as auto-antibodies, including in some cases, anti-costimulator molecules, in particular, those for Cytotoxic T-lymphocyte-Associated protein 4 ((CTLA-4) (CD152)) on CD4+ cells (Matsui et al., Autoantibodies to T Cell Costimulatory Molecules in Systemic Autoimmune Diseases 1999, J. Immunol., 162:4328).

Efforts are underway to attack cells or cellular products of the immune system and thereby treat autoimmune conditions, allergies, asthma and transplantation rejection using as reagents presumptive antigenic peptides or proteins, peptides representing certain T cells, monoclonal antibodies (Mabs) or recombinant proteins binding various effector cells or molecules such as Tumor Necrosis Factor Alpha (TNFα) and IgE.

The following immunomodulatory approaches are contrasted with the mode of action for antigen specific products. For example, a fusion protein LFA-3TIP (Amevive™ from Biogen), purportedly a molecule composed of the first extracellular domain of LFA-3 fused to the hinge (CH2 and CH3 domains of human IgG1=TIP) which targets the CD2 receptor on T cells is being evaluated for psoriasis and for xeno- and allograft rejection. LFA-3TIP is bi-functional (i.e., two identical LFA-3 regions and TIP) and therefore is a complex conjugate molecule. According to Biogen, LFA-3TIP is a recombinant fusion protein designed to modulate the immune response by blocking the cellular pathway that activates T cells. Presumably, the compound is acting on a subset of memory effector cells with a down modulation or re-direction of modulation activity.

Other antigen non-specific approaches also utilize monoclonal antibodies that act on activated T cells and down regulate them such as using anti-CD3 (Protein Design Laboratories) or blocking Antigen Presenting Cells (APC) and T cell interaction by anti-Intercellular Adhesion Molecule 3 ((ICAM-3) (ICOS)). Another such example is MEDI-507 (Medimmune) which is believed to be a humanized monoclonal antibody for psoriasis that also targets CD2 presumably for removing or inactivating those cell types. Other diseases such as tissue transplantation rejection and allergies are also being tested by this approach.

In contrast to acting on cell surface markers such as ICAM-3, peptide rhu-Mab-E25 (Genentech) binds to circulating IgE with the goal of preventing activation of mast cells. rhu-Mab-E25 is believed to be a humanized monoclonal antibody against IgE. Other researchers are developing monoclonal antibodies to act directly on agents causing disease symptoms. Remicade Infliximab (Centocor) is purported to be a monoclonal antibody to TNFα while anti CD40 ligand has been used for treatment in animal model of MS (Howard et al., supra). A recombinant generated designed protein Enbrel (Immunex) is purported to comprise two molecules of r-DNA derived TNFα soluble receptor, and is intended to block TNFα's action.

It should be noted, however, that many of these agents are not disease specific and often recognize and could disadvantageously affect normal cellular and body constituents that have a defined and necessary role in needed normal immune defenses.

One example of an antigen disease specific approach is to treat MS patients by oral administration of myelin proteins. Collagen type II for treatment of patients with Rheumatoid Arthritis can also be used. These treatments are designed to target the Gut Associated Lymphoid Tissues (GALT) to induce tolerance by antigen specific suppression of the immune system. It is not known if these treatments would use the intact protein or a hydrolyzate containing smaller peptides; Wucherpfennig et al., Shared human T cell receptor V beta usage to immunodominant regions of myelin basic protein. 1990, Science, 248:1016; Ota et al., T-cell recognition of an immunodominant myelin basic protein epitope in multiple sclerosis 1990, Nature, 346; 183).

A related approach to treat autoimmune conditions is the use of an oral formulation of peptide(s) as immunogen given in large quantities. The peptide represents a sequence that is thought to be the autoimmune epitope itself or a modified form which may have altered binding or improved stability properties. By use of the peptide it is believed that either the normal peptide or an Altered Peptide Ligand (APL) will bind to the T cell receptor (TCR) and induce a state of anergy with an antigen presenting cell (APC) (Faith et al., An Altered Peptide Ligand Specifically Inhibits Th2 Cytokine Synthesis by Abrogating TCR Signaling 1999, J. Immunol., 162:1836; Soares et al., Differential Activation of T Cells by Natural Antigen Peptide Analogues: Influence on Autoimmune and Alloimmune In Vivo T Cell Responses 1998, J. Immunol., 160:4768; Croft et al., Partial activation of naive CD4 T cells and tolerance induction in response to peptide presented by resting B cells 1997, J. Immunol., 159:3257, Ding et al., Differential Effects of CD28 Engagement and IL-12 on T Cell Activation by Altered Peptide Ligands 1998, J. Immunol., 161:6614; Hin et al., Cutting Edge: N-Hydroxy Peptides: A New Class of TCR Antagonists 1999, J. Immunol., 163:2363). Some of the approaches with APL include using related amino acids such a D amino acids (Koch et al., A Synthetic CD4-CDR3 Peptide Analog Enhances Skin Allograft Survival Across a MHC Class II Barrier 1998, J. Immunol., 161:421), amino acids with substituted side chains (Palma et al., Use of Antagonist Peptides to Inhibit In Vitro T Cell Responses to Par j1, the Major Allergen of Parietaria judaica Pollen 1999, J. Immunol., 162:1982), methylene groups to replace peptide bonds in the peptide backbone (Meda et al., Beta-amyloid (25-35) peptide and IFN-gamma synergistically induce the production of the chemotactic cytokine MCP-1/JE in monocytes and microglial cells 1996, J. Immunol., 157:1213) and N-hydroxyl peptides (Hin et al., supra).

Various substitutions of side chains of the Major Histocompatibility Complex (MHC) and T cell receptor (TCR) molecules have also been contemplated by the prior art (Clay et al., Changes in the Fine Specificity of gp100₍₂₀₉₋₂₁₇₎-Reactive T Cells in Patients Following Vaccination with a Peptide Modified at an HLA-A2.1 Anchor Residue 1999, J. Immunol., 162:1749). For example, with insulin activity it has been shown that a one amino acid change on the α-chain can abolish its oral immune tolerance activity in two (2) mechanistically different IDDM murine models (Homann et al., Insulin in Oral Immune “Tolerance”: A One-Amino Acid Change in the B Chain Makes the Difference 1999, J. Immunol., 163:1833). Although not an autoimmune epitope, a single change from threonine to alanine can abolish biological activity (Sutherland et al., An 11-Amino Acid Sequence in the Cytoplasmic Domain of CD40 Is Sufficient for Activation of c-Jun N-Terminal Kinase, Activation of MAPKAP Kinase-2, Phosphorylation of ImBc, and Protection of WEHI-231 Cells from Anti-IgM-Induced Growth Arrest 1999, J. Immunol., 162:4720) while a switch from phenylalanine to alanine converts the bee venom phospholipase to an inactive form (Faith et al., supra) as does a switch from tyrosine to alanine convert from active to inactive for another system (Hausman et al., Peptide Recognition by Two HLA-A2/Tax₁₁₋₁₉-Specific T Cell Clones in Relationship to Their MHC/Peptide/TCR Crystal Structures 1999, J. Immunol., 162:5389).

In yet another approach based on peptide materials, truncated peptides of autoimmune inducing epitope are used as an antagonist in an animal model to treat a particular condition (Karin et al. supra). Synthetic amino acid polymers that are thought to represent epitopes which contain Tyrosine (Y), Glutamic acid (E), alanine (A) and lysine (K) to target T cells such as Copolymer 1 have been contemplated. For example, Copaxone has been used as an oral tolerance delivery approach to treat MS patients where Copaxone is believed to be a synthetic copolymer of four amino acids (Hafler et al., supra 1988, J. Immunol., 141:131). Modified peptides of peptide epitopes are also being studied for treatment of various autoimmune conditions including MS and PV (desmoglein-3) (Hammer, et al., supra 1997, Adv. Immunol., 66:67; Wucherpfennig et al., Structural Basis for Major Histocompatibility Complex (MHC)-Linked Susceptibility to Autoimmunity: Charged Residues of a Single MHC Binding Pocket Confer Selective Presentation of Self-Peptides in Pemphigus Vulgaris 1995, PNAS, 92:11935). The use of myelin proteolipid associated peptide epitope, a polymer or derivative of this epitope for MS has been further contemplated (Hammer et al., supra 1997, Adv. Immunol., 66:67).

In other approaches, peptides that are unique to the T cell antigen receptor molecule have been contemplated for a psoriasis vaccine such as IR 502 while others have been contemplated for Rheumatoid Arthritis. These peptides are found in a particular part of the variable region usually the third hyper-variable region of the beta chain of the T cell antigen Receptor (TCRαVX) (Kotzin et al., Preferential T-Cell Receptor β-Chain Variable Gene Use in Myelin Basic Protein-Reactive T-Cell Clones from Patients with Multiple Sclerosis 1991, Proc Nat Acad Ssi US, 88:9161; Oksenberg, et al., Limited heterogeneity of rearranged T-cell receptor V alpha transcripts in brains of multiple sclerosis patients. 1990, Nature, 345:344). For example, in an immune response to TCRαV3 (Sutherland et al., supra 1999, J. Immunol., 162:4720) a peptide is generated whose objective is to eliminate particular T cells, and by removing the T cells responsible for the condition, treat the underlying condition. However, this approach has the disadvantageous potential of eliminating other T cells that contain the same αV3 peptide sequence besides the one responsible for the autoimmune condition.

Still another peptide approach uses complimentary peptide vaccine that induces T cell anergy and prevents EAE in rats by induction of anti-TCR antibodies (a la anti-idiotype) and thereby elimination of these cells (Araga et al., supra 1999, J. Immunol., 163:476).

For some diseases the appearance of such modified self antigens is part of the disease process, the approach may be completely different. For example in Alzheimer's Dementia (AD), the normally cleared amyloid beta (Aβ) protein fragments are deposited in AD plaques wherein the desired response is to slow down, arrest or even reverse deposition and the subsequent pathological effects.

To accomplish this therapeutic approach, two basic immunological approaches have been evaluated requiring the use of so called “active” and/or “passive” agents.

Generally, “passive agents” as known in the art are composed of Mabs targeting cytokines, or cell surface marker, or soluble receptor with similar binding specificity. However, a problem encountered with “passive agents” is that they must be frequently administered parenterally for the life of the patient. Moreover, the Mabs or soluble receptor must be “humanized” (huMab) to allow long term administration and to avoid potential long term adverse effects. Hence, the production and sale of Mabs comprises one of the largest markets for treating chronic long-term conditions in the developed world.

For autoimmune uses, the use of agents for treatment ranges from insulin for type 1 diabetes to modern biotechnology treatments related to monoclonal antibodies and soluble receptors to complement cytokines and/or cell surface markers. Well known examples of modern biotechnology treatments include Remicade®, Enbrel® Humira® for rheumatoid arthritis, Amieve® and several interferon-β products for MS. Other examples of Mabs include materials used in clinical trials for psoriasis and MS. However, these products only act upon the major abnormal amounts of molecule such as TNF-α, complement, or cells with a particular surface marker and fail to treat the underlying condition.

For the treatment of AD, AD Mabs are being investigated as potential products by a number of private corporations. Notably, one known Aβ Mab investigated by Lilly is effective in decreasing Aβ burden in the cerebral cortex of “AD” mice. This finding confirms previous reports of beneficial effects of plaque reduction in “AD” transgenic mice by other known Ab both Mab and Pab investigated by Elan (Bard et al., Epitope and isotype specificities of antibodies to beta-amyloid peptide for protection against Alzheimer's disease-like neuropathology, Proc Natl Acad Sci U.S.A. 2003 Feb. 18; 100(4):2023-8; DeMattos et al. Peripheral anti-A beta antibody alters CNS and plasma A beta clearance and decreases brain A beta burden in a mouse model of Alzheimer's disease, Proc Natl Acad Sci U.S.A. 2001 July 17; 98(15):8850-5).

However, it should be noted that not all of the known Mab anti-Aβ being tested were shown to be effective (Bard et al. supra). Differences have been found in the ability of the respective antibodies to pass the Blood Brain Barrier (BBB). Clearly, the mechanism by which Aβ plaques are reduced is not understood.

For example, the necessity for inclusion of the Fc region was shown to be required in one compound entitled 3D3 which was found in the Central Nervous System (CNS) (Bard et al.). The Mab 3D3 was mediated by binding to phagocytes. It was shown by the art that 3D3 (IgG2b) and 10D5 (IgG1) were effective in reducing Aβ plaques but not 16C11 (IgG1) and 21F12 (IgG2a). Both IgG2a and IgG2b subclasses are considered as complement fixing antibodies that bind to Fc receptors. Furthermore, an association between Aβ plaque reduction and affinity of the Mabs was not validated. Further, Mab m266, is believed to act on CNS Aβ levels by serving as a sequestering agent acting from its plasma location given that it is not found in the CNS (DeMattos et al). It appears from the art that m266, 3D3 and 10D5 act by different mechanisms in plaque clearance wherein all three Mabs act as Aβ sinks in dialysis assays.

With regard to “active agents” in the treatment of Aβ, the objective is to induce the body to produce molecules to counteract or neutralize the agent. Hence, “active agents” can be considered vaccines that induce a long lived adaptative immune response in patients through induction of antibodies and/or by Cytotoxic Lymphocytes (CTL) or other cellular mechanisms to clear or impede the deposition of Aβ. Several reports have shown induction of immune responses having effects on plaque formation in murine AD models (Schenk et al. 1999 Immunization with amyloid-beta attenuates Alzheimer-disease-like pathology in the PDAPP mouse Nature 400: 173, Sigurdsson et al. 2001 Immunization with a nontoxic/nonfibrillar amyloid-beta homologous peptide reduces Alzheimer's disease-associated pathology in transgenic mice Am J Pathol 159:439).

The role of antibodies has been implicated in both the “passive” and the “active” immunization approach. However, the role of CTL or other cellular responses is less certain. For example, antibody alone may be sufficient for plaque clearance (Schenk et al., supra).

In spite of the state of the art with respect to recombinant proteins and their use as therapeutics, only one recombinant protein based vaccine is available as an approved successful vaccine, for Hepatitis B surface protein (HBsAg) however several sources are available including vaccines from Merck, GSK, Sanofi. Recombinant proteins for HSV, several other viruses, Chylamdia, Lyme disease have all not been commercially successful or failed to meet approvable criteria and workers using them are now looking for enhancers such as cytokines, new adjuvants etc.

Major problems are also associated with viral vector vaccines. The primary problem is the high immune response induced against the vector itself. This induced immune response severely limits the number and frequency of subsequent injections/boosters that can be administered. Moreover, some adenoviruses have the potential for causing allergic conditions such as celiac disease. It is also known that many viral proteins, including some from HIV and HSV contain immunosuppressive epitopes. Viral proteins are also suspected as causative agents for other autoimmune conditions such as type 1 diabetes, Multiple Sclerosis (MS), Myocarditis, and Graves disease.

Similarly, virus like particles (VLP) also suffer from the same disadvantages noted for viral vector vaccines whereby the particles incorporate antigenic epitope(s) of the plant, animal or bacterial virus (plasmid) where it is expressed in the VLP containing the foreign antigen of interest perhaps in a fusion protein along with the epitopes of the host virus or plasmid.

Another disadvantage of using a DNA-based vaccine for autoimmune conditions is the possibility of the vaccine DNA being integrated into the host's genome. One alternative is to conjugate a particular epitope to a carrier protein to avoid such incorporation into the genome. It is known within the art to use large carrier proteins such as Keyhole Limpet Hemocyanin (KLH), Bovine Serum Albumin (BSA), or Antigenics' heat shock proteins (HSP) coupled/conjugated or incorporated with a virus protein.

Other options for peptide delivery of peptide epitopes include the use of synthetic biodegradable microparticles like Poly (lactide-co-glycolide) PLG with aggregated antigen. Still other delivery technologies for peptide antigens include AutoVac™ of Pharmexa, Corixa's LEif™, Therion Biologic's or various other Virus Like Particle (VLP) technologies. Other small molecule delivery technologies for peptides are Antigen Express's ‘Ii-Key’ delivery, phage display and Multiple Antigen Presentation (MAPS) technologies (Rosenthal 2005 Immune peptide enhancement of peptide based vaccines Frontiers in Bioscience 1:478-482).

But again, all the known approaches have the major disadvantage of using large, very immunogenic particles. Moreover, patient populations requiring such therapeutics have usually been exposed to many of these same antigens during their lifetimes. Hence, and similar to vaccine vector delivery of antigens, clearance of the antigen can be so vigorous in the previously exposed host that no response will occur to the new antigen. On the other hand, a strong immune response may occur upon reintroduction of the vector. For example, in the case of the conjugate VP22 containing HSV-1 protein, the response may be undesirable given that a majority of adults have had one or more exposures to HSV-1 (Muran-yiova et al. 1991 Immunoprecipitation of herpes simplex virus polypeptides with human sera is related to their ELISA titre. Acta Virol. 35:252-9).

Hence, it is critical that the therapeutic be a small peptide used as a potential antigen as in Epimmune's Padre™ or CEL-SCI's Ligand Epitope Antigen Presenting System (L.E.A.P.S.™) technology as described in U.S. Pat. No. 5,652,342, the contents of which are incorporated herein by reference.

L.E.A.P.S. uses small peptides referred to as T cell Binding Ligands (TCBL's) as a peptide antigen delivery technology wherein the TCBL's are peptide sequences derived from the human immune system molecules known or suspected to bind to human T cells. Although L.E.A.P.S. includes a first peptide which is an antigenic peptide associated with disease or the causative organism of disease covalently bonded to a second peptide which is a T cell binding ligand, the hetero-functional cellular immunological reagents taught by U.S. Pat. No. 5,652,342 are not antigen (disease) specific in certain cases. Instead, U.S. Pat. No. 5,652,342 teaches T cell binding ligands including portions of MHC Classes I and II or accessory molecules such as 0-2-microglobulin, portions of LFA-3, portions of the Fc region of the heavy chain of immunoglobulins, and Ia⁺ molecules and generally teaches for the antigens associated with auto-immunity such as IDDM, RA and Thyroiditis. U.S. Pat. No. 5,652,342 fails to provide more antigen (disease) specific treatment.

Hence, there is a need for approaches to treating autoimmune diseases with immunotherapeutics requiring more specific and lower doses of active substance and less frequent dosing. The therapeutics should also allow for the option of subcutaneous, oral intradermal, intranasal or transdermal administration wherein the active portion of the therapeutics should be comprised of substantially smaller molecules (3-5000 Da) that are more likely to be able to cross the BBB.

Moreover, the need extends to treatments that do not require monoclonal antibodies or receptor agonists to molecules that are critical to maintaining normal body function such as TNF-α Soluble Receptor or those that require “humanization”.

There is further a need for therapeutics that does not eliminate essential or necessary T cells other than those responsible for the underlying disease. The therapeutics should only act upon the major abnormal molecule or cell while preventing or inducing a high response against the vector or delivery molecule.

There is further a need for a therapeutic that is not integrated into the host's genome wherein it may alter other normal or onco genes, their expression or activation.

There is still further a need for providing specific antigenic peptides associated with disease or the causative organism that can be linked with a T cell binding ligand such as L.E.A.P.S. Moreover, the therapeutics should be manufactured and be cost effective in its production and stable during storage before use.

### BRIEF SUMMARY OF THE INVENTION

The present invention provides peptide constructs useful for treatment of autoimmune disease such as multiple sclerosis, pemphigus vulgaris, or other conditions such as asthma, allergy, diabetes, and tissue transplantation rejection (including both host-versus-graft and graft-versus-host rejection), which differ from the above approaches used with antigenic peptide alone. The novel constructs bind in an antigen specific manner and redirect the T cell in the direction of a non-deleterious autoimmune response, primarily from a Th1 to a Th2 immune response, but where advantageous, primarily from a Th2 to a Th1 immune response. Alternatively, the novel constructs include one peptide component which will bind to T cells associated with autoimmune disease, asthma, allergies or host versus graft or graft versus host rejection while a second peptide component will bind to sites on the T cells which will preclude the normal sequence of events required for cell activation thereby initiating an abortative T cell modulation resulting in cell anergy and apoptosis.

Specifically, the novel peptides of this invention include peptide constructs of the following formula (I):
P₁-x-P₂ (I)
where P₁ is a peptide associated with autoimmune disease, allergy, asthma, host-versus-graft rejection, myocarditis, diabetes, tissue transplantation rejection or immune mediated disease and which will bind to an antigen receptor on a set or subset of T cells; P₂ is an immune response modifying peptide which will (i) cause a directed immune response by said set or subset of T cells to which the peptide P₁ is attached or (ii) bind to a T cell receptor which will cause said set or subset of T cells to which the peptide P₁ is attached to initiate, but not complete, an immune response causing said set or subset of T cells to undergo anergy and apoptosis; and x is a direct bond or linker for covalently bonding P₁ and P₂.

Alternatively, the invention contemplates a variable immunomodulatory peptide construct having the formula (II)
P₃-x-P₄

where P₃ is a peptide construct comprised of X₁ to X₁₄ said peptide P₃ being associated with autoimmune disease, allergy, asthma, host-versus-graft rejection, myocarditis, diabetes, and immune-mediated disease, which binds to an antigen receptor on a set or subset of T cells, and

P₄ is a peptide construct comprised of X₁ to X₁₄ causing a Tₕ2 directed immune response by said set or subset of T cells to which the peptide P₃ is attached or which binds to a T cell receptor causing said set or subset of T cells to which the peptide P₃ is attached to initiate, but not complete, an immune response causing said set or subset of T cells to undergo anergy and apoptosis;

such that X₁ to X₁₀ and X₁₄ describe a group of amino acids based on their features and X₁₁ to X₁₃ describe modifications to the peptide construct, wherein

X₁ is selected from the group consisting of Ala and Gly,

X₂ is selected from the group consisting of Asp and Glu,

X₃ is selected from the group consisting of Ile, Leu and Val,

X₄ is selected from the group consisting of Lys, Arg and His,

X₅ is selected from the group consisting of Cys and Ser,

X₆ is selected from the group consisting of Phe, Trp and Tyr,

X₇ is selected from the group consisting of Phe and Pro,

X₈ is selected from the group consisting of Met and Nle,

X₉ is selected from the group consisting of Asn and Gln,

X₁₀ is selected from the group consisting of Thr and Ser,

X₁₁ is Gaba^{χ} where X₂X₃, X₃X₂, X₂X₃, X₃X₂, X₃X₃, or X₂X₂ can be substituted with X₁₁;

X₁₂ is selected from the group consisting of acetyl, propionyl group, D glycine, D alanine and cyclohexylalanine;

X₁₃ is 5-aminopentanoic where any combination of 3 to 4 amino acids of X₂ and X₃ can be replaced with X₁₃;

X₁₄ is selected from the group consisting of X₁, X₂, X₃, X₄, X₅, X₆, X₇, X₈, X₉ and X₁₀; and

x is a direct bond or linker for covalently bonding P₃ and P₄;

The present invention also provides a first method for treating or preventing inappropriate autoimmune response in individuals at risk for autoimmune disease, allergic reactions, asthma or host-graft or graft-host rejection, wherein a pharmacologically effective amount of a peptide construct of formula (I) is administered to the individual to effectively eliminate the set or subset of T cells involved in the autoimmune response.

The present invention also provides a second method for modulating an inappropriate autoimmune response in individuals at risk for autoimmune disease, allergic reactions, asthma or host-graft or graft-host rejection, wherein a pharmacologically effective amount of a peptide construct of formula (I) is administered to the individual to redirect the autoimmune response from a Th1 to a Th2 immune response, or from a Th2 to a Th1 immune response, whereby the inappropriate autoimmune response is modulated to decrease or eliminate the adverse effects associated with the inappropriate autoimmune response.

### DETAILED DESCRIPTION OF THE INVENTION

It has been reported that the, failure of mature CD8 cells to simultaneously engage their TCR and CD8 co-receptor triggers an activation process that begins with inhibition of CD8 gene expression through remethylation and concludes with up-regulation of surface Fas and Fas ligand and cellular apoptosis (Pestano et al., Inactivation of Misselected CD8 T Cells by CD8 Gene Methylation and Cell Death 1999, Science, 284:1187). This is consistent with the results of others where if full engagement of certain very major coreceptors are not effected then an activation process, but abortative in nature, leading to apoptosis occurs (see also Gogolak et al., Collaboration of TCR-, CD4- and CD28-mediated signaling in antigen-specific MHC class II-restricted T-cells 1996, Immunol. Let. 54:135; Grakoui et al., The Immunological Synapse: A Molecular Machine Controlling T Cell Activation 1999, Science, 285:221; Malissen, Dancing the Immunological Two-Step 1999, Science, 285:207; Redpath et al., Cutting Edge: Trimolecular Interaction of TCR with MHC Class II and Bacterial Superantigen Shows a Similar Affinity to MHC:Peptide Ligands 1999, J. Immunol., 163:6; Preckel et al., supra 1998, Eur. J. Immunol., 28:3706; Sambhara et al., Programmed cell death of T cells signaled by the T cell receptor and the alpha 3 domain of class I MHC 1991, Science, 252:1424; Kishimoto et al., Strong TCR Ligation Without Costimulation Causes Rapid Onset of Fas-Dependent Apoptosis of Naive Murine CD4⁺ T Cells 1999, J. Immunol., 163:1817; Kubo et al., CD28 Costimulation Accelerates IL-4 Receptor Sensitivity and IL-4-Mediated Th2 Differentiation 1999, J. Immunol., 163:2432).

Therefore, a different approach would be to have a modulation but not with a full sequence of events, the construct binding in an antigen specific manner with the antigenic epitope but the TCBL ligand binding to a site on another molecule associated with certain early events that are early intermediates in the full expression pathway thereby occupying the space and causing an early event in the process of activation (such as Ca⁺⁺ flux, activation of various phosphatases, membrane migration events, such as “patching” or “capping”, changes in RNA metabolism) but not supporting the complete activation process which can be thought of as culminating in antigen specific antibody or non-antibody mediated specific Cytotoxic T Lymphocyte activity such as killing of infected or tumor cells, by acting on DNA synthesis, cell division, or cytokine secretion, namely, without allowing the ultimate tertiary complex of binding events (MHC, antigen TCR and CD4 (or CD8)) necessary for full activation by being out of the normal temporal sequence of events. Perhaps this early binding would be of such strength that it does not disassociate and allow the cell surface rearrangement necessary for the full and normal sequence of modulatory events, such as, proliferation or secretion of late cytokines such as Fas, TNF-α or IFN-γ and thereby prohibiting events found in an autoimmune disease associated pathway with complete T cell activation. For example, initially after antigen binding to the TCR ICAM-1 (also known as CD54) on APC binding to a T cell's LFA-1 (also known as CD11a/CD18) there is a shifting away and a rearrangement with clustering of the MHC and antigenic peptides on APC binding eventually by migration on T cell membrane to a clustering of TC and CD4 (or CD8) (Malissen supra 1999, Science, 285:207; Grakoui et al., supra 1999, Science, 285:221).

According to one embodiment of this invention, such rearrangement is prevented by the close association in a peptide construct using a TCBL from ICAM-1, LFA-3 (aa26-42), VLWKKQKDKVAELENSE (SEQ ID NO.4) (Osborn et al., Amino acid residues required for binding of lymphocyte function-associated antigen 3 (CD58) to its counter-receptor CD2 1995, JEM, 181:429), by either the disparity in the temporal binding or higher strength of binding activity, thereby preventing the rearrangements and other more intimate interactions necessary for activation. Initially these sites are close together but normally rearrangements on the T cell surface occur during the activation process so by preventing this shift activation should not occur. Likewise, a TCBL from CD4 that binds to the TCR and CD3 may be used as the TCBL in the peptide construct of this invention. Its binding to the T cell recognition site will inhibit subsequent events from occurring (MHC II with CD4 or β-2 with CD8).

Still another approach is a construct which redirects the immune response initiated by the natural autoimmune inducing event from a TH1 to a TH2 response (e.g., Lowrie et al., Therapy of tuberculosis in mice by DNA vaccination 1999, Nature, 400:269; Tisch et al., supra 1999, J. Immunol., 163:1178). As used herein, a TH2 directed response is one which directs the immune response toward the TH2 direction, thus favoring production of more cytokines IL-5, IL-4, IL-10, IL-13 TNF-a and antibody isotypes IgG1 and IgG3 in mice (or comparables in man) as opposed to Th1, where the immune response favors production of cytokines IFN-γ, IL-2, IL-6, IL-12 cytokines and antibody isotypes IgG2a and IgG2b in mice and Cytotoxic T cell activity. It is understood, of course, that a “TH2 directed response” is not intended to imply an exclusively TH2 response, but rather a mixed immune response which is weighted to favor a TH2 profile.

According to this embodiment a TCBL associated with TH2 responses; e.g., peptide G from MHC class II (Zimmerman et al., A new approach to T cell activation: natural and synthetic conjugates capable of activating T cells 1996, Vacc. Res., 5:91, 5:102; Rosenthal et al., 1999, Vaccine), IL-4 or IL-5 or peptides known to stimulate IL-4 or IL-5 synthesis are used as the TCBL along with the autoimmune inducing peptide (e.g., Hammer et al., supra, Krco et al., supra, Araga et al., supra, Ota et al., supra, Ruiz et al., supra, Yoon et al., supra, Dittel et al., Presentation of the Self Antigen Myelin Basic Protein by Dendritic Cells Leads to Experimental Autoimmune Encephalomyelitis 1999, J. Immunol., 163:32; Gautam et al., A Viral Peptide with Limited Homology to a Self Peptide Can Induce Clinical Signs of Experimental Autoimmune Encephalomyelitis 1998, J. Immunol., 161:60, the disclosures of which are incorporated herein by reference thereto) in the peptide conjugate. These peptide constructs may be used, for example, to treat type I diabetes. In an animal model the mechanism of diabetes prevention in the RIP-NP model was shown to be mediated by insulin 1-chain, and IL-4 producing regulatory cells acting as bystander suppressors (Homann et al., supra 1999, J. Immunol., 163:1833). Such redirection of immune responses have been previously reported by a DNA vaccine for TB which redirected the immune response from an inefficient response TH2 to a response that was a very effective Th1 (Lowrie et al., supra 1999, Nature, 400:269). Thus, redirecting an already existing immune response from a TH1 to a TH2 would be effective for treating autoimmune related diseases. A TCBL involved in CD28 costimulation (Kubo et al., supra) could also be effective for this purpose. If, on the other hand, the need was to redirect from a TH2 to a TH1, much less likely to be needed since many autoimmune conditions are thought to be the manifestation of deleterious TH1 effects, then a TCBL such as peptide J, DLLKNGERIEKVE (SEQ ID NO. 51) (Zimmerman et al., supra; Rosenthal et al., supra) or ones known to stimulate IL-2 or IL-12 synthesis would be used along with the autoimmune inducing peptide.

Yet another approach is to use the peptide construct to not activate the normal immune process but to activate the process leading to apoptosis of the T cell by using as the TCBL a ligand that binds to a site on the T cell whose normal binding and activation leads to apoptosis of the T cell; such as the TNF-receptor of the T cell, in which the TCBL would be the TNF-α ligand portion. Examples of such TNF peptides known to activate macrophages are amino acids 70-80 PSTHVLITHTI (SEQ ID NO. 5) (Britton et al., 1998, I & I, 66:2122) and perhaps the antagonist peptide represented by DFLPHYKNTSLGHRP (SEQ ID NO. 6) of another region (Chirinos-Rojas et al., A Peptidomimetic Antagonist of TNF-&-Mediated Cytotoxicity Identified from a Phage-Displayed Random Peptide Library 1998, J. Immunol., 161:5621). It has been suggested in WO 99/36903A1 that the H4-1-BB ligand is useful as a treatment for autoimmune disease similar to uses for flt3-L and CD40L; therefore, H4-1BB may also be used as TCBL for inclusion with autoimmune antigens to form the inventive peptide construct. Other such TCBL examples are available from application with Fas and Fas-ligand including the noncleavable Fas-ligand (WO 99/36079A1).

Since autoimmune reactive cells in disease are most likely already activated and may be expressing Fas (Tomita et al., Tetrapeptide DEVD-aldehyde or YVAD-chloromethylketone inhibits Fas/Apo-1(CD95)-mediated apoptosis in renal-cell-cancer cells 1996, Int. J. Can., 68:132; Lie et al., Synthesis and biological activity of four kinds of reversed peptides 1996, Biol. Pharma. Bul. 19:1602) the Fas-Ligand or the sequence obtained by reverse engineering technique to determine amino acid (aa) sequence acting as receptor for DEVD-aldehyde or YVAD (SEQ ID NO. 22) chloromethylketone, may also be used as the TCBL. Representatives from another pair, IFN-γ and the IFN-γ ligand can also be used as TCBL's in the invention peptide constructs.

In this invention the antigenic peptide and the peptide for T cell binding (TCBL) may be directly linked together in any order (i.e., N-terminal of one to C-terminal of other or vice versa) or the peptide may be covalently bonded by a spacer or linker molecule. With regard to linkers between the two domains, suitable examples include a thioether bond between an amino terminus bromoacetylated peptide and a carboxyl terminus cysteine, often preceded by a diglycine sequence (Zimmerman et al., supra), carbodiimide linkages, a multiple glycine, e.g., from 3 to 6 glycines, such as triglycine, with or without one or two serines, separation between the two entities, e.g., GGGS (SEQ ID NO.7), GGGSS (SEQ ID NO. 8), GGGGS (SEQ ID NO.9), GGGGSS (SEQ ID NO. 10), GGGSGGGS (SEQ ID NO.11), etc., and other conventional linkages, such as, for example, the direct linkages such as, EDS, SPDP, and MBS, as disclosed in the aforementioned U.S. Pat. No. 5,652,342.

Thus, the peptide constructs of this invention may be conveniently represented by the following formula (I):
P₁-x-P₂ (I)

where P₁ is a peptide associated with autoimmune disease, allergy or asthma, or transplantation rejection and which will bind to an antigen receptor on a set or subset of T cells;

P₂ is an immune response modifying peptide which will bind to T cells to cause a directed immune response by said set or subset of T cells to which the peptide P₁ is attached or which will bind to a T cell receptor which will cause said set or subset of T cells to which the peptide P₁ is attached to initiate, but not complete, an immune response causing said set or subset of T cells to undergo anergy and apoptosis; and x is a direct bond or linker for covalently bonding P₁ and P₂.

The TCBL portion of the immunomodulatory peptide construct of this invention, i.e., P₂, may comprise a discontinuous epitope composed of two small regions separated by a loop or by a single chain short peptide in place of the loop (Shan et al., Characterization of scFv-Ig Constructs Generated from the Anti-CD20 mAb 1F5 Using Linker Peptides of Varying Lengths 1999, J. Immunol., 162:6589). For example, an eight amino acid group, LRGGGGSS (SEQ ID NO.12), of 11.2 Angstroms in length (Reineke et al., A synthetic mimic of a discontinuous binding site on interleukin-10 1999, Nature Biotechnology, 17:271) has been used to form a single peptide from two smaller discontinuous peptides of IL-10, thereby forming a TCBL which could be used for redirection from a TH1 to a TH2, in combination with, for example, the IDDM, PV or MS inducing epitopes (Hammer et al., supra Tisch et al., supra).

Linkers (X) of varying lengths to form a single chain may be used, for example, GGGS (SEQ ID NO. 7), GGGGS (SEQ ID NO. 9), including, from among 1 or more repeats of this tetrapeptide or pentapeptide, e.g., GGGSGGGS (SEQ ID NO. 11), GGGSGTGSGSGS (SEQ ID NO. 52). Such linkers may result in a tertiary structure which might be of use to form a more avid TCBL (Shan et al., supra).

Although not strictly limited, the peptide constructs of this invention may have as many as about 200 amino acids in its sequence, preferably up to about 150 amino acids, and especially, up to about 100 amino acids. The minimum number of amino acids is also not strictly limited but usually each of the peptide components P₁ and P₂ will have at least about 4, preferably at least about 6, and more preferably at least about 8 or 9 amino acids in order to provide the appropriate epitope configuration for effectively binding to the appropriate site on the T cells of interest. Thus, the peptide constructs of this invention will usually contain from about 20 to about 100 or more amino acids.

The peptide constructs may be prepared using conventional solid state peptide synthesis, provided however, that for constructs having more than about 40 amino acids, especially more than about 50 amino acids, it is usually convenient and preferred to prepare shorter segments and then link the shorter segments using well known techniques in solid phase peptide synthesis.

The peptide constructs may be prepared using conventional solution phase condensation chemistry of smaller peptides prepared by solid phase peptide synthesis, provided however, that for constructs having more than about 40 amino acids, especially more than about 50 amino acids, it is usually convenient and preferred to prepare shorter segments and then link the shorter segments using well known techniques in solid phase peptide synthesis.

Alternatively, the peptide constructs of this invention may be prepared using well known genetic engineering methods. Further details on methods for producing the instant peptide constructs can be found in the aforementioned U.S. Pat. No. 5,652,342.

Improved versions of the peptide constructs are comprised of variables X₁ to X₁₂ substitutions where each of X₁ to X₁₂ describe a group of particular types of amino acids based on their features. For example, it is known that amino acids can be nonpolar, hydrophobic while another group of amino acids are polar, uncharged amino acids that are hydrophilic. Still further, it is known that amino acids can be grouped as those that are polar, charged, hydrophilic amino acids further subdivided between acidic and basic amino acids. Table 1 describes the chemical and structural features along with the corresponding amino acids.

TABLE 1

| Chemical or Structural Feature | Amino Acid |
|---|---|
| Polar/hydrophilic | N, Q, S, T, K, R, H, D, E, (C, Y)* |
| Non-polar/hydrophobic | (G), A, V, L, I, P, Y, F, W, M, C |
| H-bonding | C, W, N, Q, S, T, Y, K, R, H, D, E |
| Sulfur containing | C, M |
| Charged at Neutral | D, E, (C) |
| pH Negative/acidic | |
| Charged at Neutral | K, R, (H) |
| pH Positive/basic | |
| Ionizable | D, E, H, C, Y, K, R |
| Aromatic | F, W, Y, (H, no UV absorption) |
| Aliphatic | G, A, V, L, I, P |
| Forms covalent cross- | C |
| link (disulfide bond) | |
| Cyclic | P |

*Note:
Amino acids in parentheses have the indicated character to a limited extent.

Table 2 describes the amino acids for X₁ to X₁₀ wherein the amino acids for X₁ to X₁₀ have been selected on their common chemical and structural features as shown in Table 1 with PRT/1 code for inclusion in the improved variants of the invention. Instances of X₂X₃ or X₃X₂ or X₂X₃ or X₃X₂ or X₃X₃ or X₂X₂ can be replaced by X₁₁ or by gamma amino butyric acid (GABA) wherein the common feature is that the amino function is on the γ-carbon and not the α-carbon. Modifications for increased stability at the amino terminus by use of an acetyl or propionyl group, D glycine or D alanine or use of cyclohexylalanine at the amino terminus to reduce proteolysis are contemplated by the substitution of X₁₂. As shown in the improved variants of the sequence, X₁₂ can be present or not present on the sequence. It is noted that the sequences in computer readable form do not include X₁₂. However, the presence of the modification is present for all sequences where disclosed in the instant specification. Similarly, modifications by use of 5-aminopentanoic acid for replacement of lengths of 3 or 4 amino acids of X₂ and X₃ are also contemplated by the invention and represented as X₁₃ wherein the lengths of 3 or 4 amino acids of X₂ and X₃ can be replaced by 5-aminopentanoic acid. Although X₁₃ is not an amino acid, it is used as a place holder in some SEQ ID NO.'s to indicate 5-aminopentanoic acid bonded to the adjacent or underlying amino acid for improved stability. X₁₄ is a variable selected from any of X₁, X₂, X₃, X₄, X₅, X₆, X₇, X₈, X₉, or X₁₀.

TABLE 2

| Variable | Amino Acid | Chemical or Structural Feature |
|---|---|---|
| X₁ | A and G | Aliphatic, non polar |
| X₂ | D and E for acidic | Polar acidic, H-bonding, Charged |
| | amino acids | at Neutral pH Negative, Ionizable |
| X₃ | I, L or V | Non-polar, branched, non-cyclic |
| | | alkyl hydrocarbon |
| X₄ | K, R or H for basic | Polar basic/hydrophilic, H- |
| | amino acids | bonding, Ionizable |
| X₅ | C or S | Polar charged with similar |
| | | features as described in U.S. |
| | | Pat. No. 5,019,384 |
| X₆ | F, W or Y for | Non-polar/hydrophobic, |
| | aromatic amino acids | Aromatic |
| X₇ | F or P | Non-polar/hydrophobic, Ring |
| | | Structure* |
| X₈ | M or Nle | Natural and non-natural branched |
| | (Norleucine)^{ψ} | chain |
| X₉ | N or Q for amidated | Polar/hydrophilic, H-bonding, |
| | amino acids | Equivalent sized, acid carboxylic |
| | | acid group |
| X₁₀ | T or S | Polar/hydrophilic, H-bonding, |
| | | Capable of binding non-amino |
| | | acid molecules to protein |
| X₁₁ | Gaba^{χ} for X₂ X₃ | Amino function is on γ carbon |
| | or X₃ X₂ or X₂ X₃ or | and not α carbon |
| | X₃ X₂ or X₃ X₃ or | |
| | X₂ X₂ | |
| X₁₂ | Acetyl or propionyl | Improved stability |
| | group, D glycine or | |
| | D alanine, | |
| | Cyclohexylalanine at | |
| | amino terminus | |
| X₁₃ | 5-aminopentanoic acid | Improved Stability |
| | for replacement of 3 | |
| | or 4 amino acids of | |
| | X₂ and X₃ | |
| X₁₄ | X₁, X₂, X₃, X₄, | |
| | X₅, X₆, X₇, X₈, | |
| | X₉, or X₁₀ | |

*W is not added because W is less stable than F or P as described in U.S. Pat. No. 5,109,384
^{ψ}isomeric to leucine and isoleucine but not found in proteins and formed in the deamination of lysine. Also called caprine.
^{χ}Gamma amino butyric acid

The novel peptide constructs of this invention can also be used to treat transplant recipients, for example, for heart, liver, lung and kidney, who are either undergoing or at risk for rejection of the transplant (Host versus Graft or HvG). In this case, the peptide antigen(s) used to make the construct would be the major epitope(s) of the graft combined with a TCBL as described above to either suppress or redirect the immune response. As one of the major sources of antigenic materials causing the HvG are thought to be the MHC proteins and, in particular, the polymorphic regions that are from the graft tissue and recognized by the host's MHC, peptides from those regions of the graft's MHC molecules would be likely candidates for the antigenic epitope portion. Other regions of potential usefulness include polymorphic regions of the minor histocompatability antigens. TCBL's can be selected from, but are not restricted to the following LFA-3 or FasL described above (Lie et al., or Tomita et al., supra). Peptide G (SEQ ID NO. 15) from the MHC II molecule, or Hu IL-10 (SEQ ID NO.28) (Gesser et al., Identification of functional domains on human interleukin 10 1997, Proc. Nat. Acad. Sci. 94:14620; Lie et al., supra and Tomita et al., supra) may be selected for redirection of immune responses.

As activated T cells normally express MHC molecules, another way of immunomodulation is to take advantage of the programmed pathway established by antigen addition. T cells which receive a signal from the TCR and the MHC I to CD-8⁺ cells undergo apoptosis without other costimulatory signals (Sambhara et al., supra 1991, Science, 252:1424). Therefore, the TCBL, peptide E, (the α3 domain amino acids 223-229 of the human MHC I conserved region can be used along with the autoimmune epitope to form a peptide construct according to another embodiment of this invention.

The peptide constructs of this invention may be used as or in vaccines as therapeutic agents for treatment of autoimmune disease or HvG. The vaccines will be administered often but not always with an adjuvant and on a regular regimen such as weekly, biweekly, monthly, quarterly, semi-annually or annually by one of the following routes, ID, IM or Sub-cu and perhaps also as a cutaneous transdermal or nasal delivery vehicle in amounts of from 1-100, usually 10-50, micrograms per kilogram of body weight.

If a binding site is known on the target T cell with a defined amino acid sequence and the TCBL amino acid sequence on the ligand is not known then determination of the DNA encoding for the peptide would allow for determination of the cDNA and thus the complementary peptide sequence using the technique of Lie et al., supra, for example. In general, the TCBL's (P₂) used to form the peptide constructs of this invention will be selected from those for normal induction and modulation of immune responses, including those selected to effect redirection from Th1 or Th2, including, for example, those that are known to be related and involved in the normal events of activation, namely, IL-2, IL-10, IL-12, IL-4, IL-1β 163-171 (VQGEESNDK (SEQ ID NO.13)) (e.g., Bajpai et al., Immunomodulating activity of analogs of noninflammatory fragment 163-171 of human interleukin-1beta 1998 Immunopharmacology, 38:237; Beckers et al., Increasing the immunogenicity of protein antigens through the genetic insertion of VQGEESNDK sequence of human IL-1 beta into their sequence 1993, J. Immunol., 151:1757; Fresca et al., In vivo restoration of T cell functions by human IL-1 beta or its 163-171 nonapeptide in immunodepressed mice 1988, J. Immunol., 141:2651; Antoni, et al., A short synthetic peptide fragment of human interleukin 1 with immunostimulatory but not inflammatory activity 1986, J. Immunol, 137:3201) and most likely derived from the final complexes (MHC-I or II and CD8 or CD4) TCR and antigenic peptide. Examples of TCBL's that are associated with earlier activation events, include for example, Fas and FasL, TNF-α and TNF-αR and those for formation of early intermediate complexes and LFA-3 and CD2.

Examples of antigens associated with autoimmune disease, allergy, asthma, and transplantation rejection, include, for example, those mentioned above, including the background discussion and those shown in the following, non-limiting, representative examples, as well as in the literature references cited herein, the disclosures of which are incorporated herein, in their entirety, by reference thereto. Additional examples, may also be found in the following literature (de Lalla et al., Cutting Edge: Identification of Novel T Cell Epitopes in Lol p5a by Computational Prediction 1999, J. Immunol., 163:1725 (Lol p5a allergen from rye grass); Gautam et al., supra 1998, J. Immunol., 161:60); as well as many others available to one of ordinary skill in the art.

In one aspect, the invention provides methods of preventing or treating a disease characterized by amyloid deposition in a patient. Such methods entail inducing an immune response against a peptide component of an amyloid deposit in the patient. Such induction can be active by administration of an immunogen or passive by administration of an antibody or an active fragment or derivative of the antibody conjugated to the TCBL epitopes contemplated by the present invention.

In some patients, the amyloid deposit is aggregated Aβ peptide and the disease is Alzheimer's disease or some other autoimmune disease. In some methods, the patient is asymptomatic. In some methods, the patient is under 50 years of age. In some methods, the patient has inherited risk factors indicating susceptibility to Alzheimer's disease. Such risk factors include variant alleles in presenilin gene PS1 or PS2 and variant forms of APP. In other methods, the patient has no known risk factors for Alzheimer's disease.

For treatment of patients suffering from Alzheimer's disease, one treatment regime entails administering a dose of Aβ peptide conjugated to the TCBL epitopes contemplated by the present invention to the patient to induce the immune response. In some methods, the Aβ peptide conjugated to the TCBL epitopes contemplated by the present invention is administered with an adjuvant that enhances the immune response to the Aβ peptide. In some methods, the adjuvant is alum. In some methods, the adjuvant is MPL.

The therapeutic agent is typically administered orally, intranasally, intradermally, subcutaneously, intramuscularly, topically or intravenously. In some methods, the patient is monitored followed administration to assess the immune response. If the monitoring indicates a reduction of the immune response over time, the patient can be given one or more further doses of the agent.

In another aspect, the invention provides pharmaceutical compositions comprising Aβ conjugated to the TCBL epitope and an excipient suitable for oral and other routes of administration. The invention also provides pharmaceutical compositions comprising an agent effective to induce an immunogenic response against Aβ in a patient, and a pharmaceutically acceptable adjuvant. In some compositions, the adjuvant comprises an oil-in-water emulsion.

The invention further provides methods of preventing or treating Alzheimer's disease. In such methods, an effective dose of Aβ peptide is administered to a patient. The invention further provides for the use of Aβ, or an antibody thereto, in the manufacture of a medicament for prevention or treatment of Alzheimer's disease.

In another aspect, the invention provides methods of assessing efficacy of an Alzheimer's treatment method in a patient. In these methods, a baseline amount of antibody specific for Aβ peptide is determined in a tissue sample from the patient before treatment with an agent. An amount of antibody specific for Aβ peptide in the tissue sample from the patient after treatment with the agent is compared to the baseline amount of Aβ peptide-specific antibody. In others methods of assessing efficacy of an Alzheimer's treatment method in a patient, a baseline amount of antibody specific for Aβ peptide in a tissue sample from a patient before treatment with an agent is determined. An amount of antibody specific for Aβ peptide in the tissue sample from the subject after treatment with the agent is compared to the baseline amount of Aβ peptide-specific antibody. A reduction or lack of significant difference between the amounts of Aβ peptide-specific antibody or Aβ peptide measured after the treatment compared to the baseline amount indicates a negative treatment outcome.

In other methods of assessing efficacy of an Alzheimer's disease treatment method in a patient a control amount of antibody specific for Aβ peptide is determined in tissue samples from a control population. An amount of antibody specific for Aβ peptide in a tissue sample from the patient after administering an agent is compared to the control amount of Aβ peptide-specific antibody. An amount of Aβ peptide-specific antibody measured after the treatment that is significantly greater than the control amount of Aβ peptide-specific antibody indicates a positive treatment outcome.

The invention further provides diagnostic kits for performing the above methods. Such kits typically include a reagent that specifically binds to antibodies to Aβ or which stimulates proliferation of T-cells reactive with Aβ.

P1 from Alzheimer or Other Brain Related Epitopes

Antigen(s) such as Amyloid Precursor Protein (APP) and its presence have a potential role in plaques as well as in uses for diagnosis and treatment of Alzheimer's patients.

APP or an amino terminus peptide AD is known to have used a 40-42 amino acid protein or subcomponents thereof are the immunogen. However, there is a need for a delivery device because smaller peptides as Aβ₁₋₄₂ shown as SEQ ID NO. 53 are not very immunogenic.

(SEQ ID NO. 53)

DAEFRHDSGYEVHHQKLVFFAEDVGSNKGAIIGLMVGGVV

Variants of Aβ₁₋₄₂ are shown as SEQ ID NO. 54 but also are not very immunogenic.

(SEQ ID NO. 54)

X₂X₁X₂X₆X₄X₄X₂X₁₀X₁X₆X₂X₃X₄X₄X₉X₄X₃X₃X₆X₆X₁X₂X₂X₃
X₁X₅X₉X₄X₁X₁X₃X₃X₁X₃X₈X₃X₁X₁X₃X₃
or
X₁₂X₂X₁X₂X₆X₄X₄X₂X₁₀X₁X₆X₂X₃X₄X₄X₉X₄X₃X₃X₆X₆X₁X₂
X₂X₃X₁X₅X₉X₄X₁X₁X₃X₃X₁X₃X₈X₃X₁X₁X₃X₃
or
X₁₂X₂X₁X₂X₆X₄X₄X₂X₁₀X₁X₆X₁₁X₄X₄X₉X₄X₁₁X₆X₆X₁X₁₁X₁
X₅X₉X₄X₁X₁X₁₁X₁X₃X₈X₃X₁X₁X₁₁
or
X₁₂X₂X₁X₂X₆X₄X₄X₂X₁₀X₁X₆X₁₁X₄X₄X₉X₄X₁₁X₆X₆X₁X₁3X₁
X₅X₉X₄X₁X₁X₁₁X₁X₃X₈X₃X₁X₁X₁₁

If immunogenic improvements are needed because of suboptimal delivery of epitopes, one known method is to use the KLH conjugate of a 28 amino acid peptide of Aβ₁₋₂₈ (SEQ ID NO. 55) described in U.S. Pat. No. 4,666,829, the contents of which are incorporated herein by reference.

DAEFRHDSGYEVHHQKLVFFAENVGSNK (SEQ ID NO. 55)

Variants of SEQ ID NO. 58 of Aβ₁₋₂₈ are shown as SEQ ID NO. 56.

(SEQ ID NO. 56)

X₂X₁X₂X₆X₄X₄X₂X₁₀X₁X₆X₂X₃X₄X₄X₉X₄X₃X₃X₆X₆X₁X₂X₂X₃
X₁X₅X₉X₄
or
X₁₂X₂X₁X₂X₆X₄X₄X₂X₁₀X₁X₆X₂X₃X₄X₄X₉X₄X₃X₃X₆X₆X₁X₂X₂
X₃X₁X₅X₉X₄
or
X₁₂X₂X₁X₂X₆X₄X₄X₂X₁₀X₁X₆X₁₁X₄X₄X₉X₄X₁₁X₆X₆X₁X₁₁X₁
X₅X₉X₄
or
X₁₂X₂X₁X₂X₆X₄X₄X₂X₁₀X₁X₆X₁₁X₄X₄X₉X₄X₁₁X₆X₆X₁X₁3X₁
X₅X₉X₄

A shorter peptide than the 40-42 peptide of the 30 amino acid form of Aβ₁₋₃₀ polylysine (6 mer) shown as SEQ ID NO. 57 can be used (Sigurdsson et al., Immunization with a Nontoxic/Nonfibrillar Amyloid-β Homologous Peptide Reduces Alzheimer's Disease-Associated Pathology in Transgenic Mice 2001 Am J Pathol 159:439).

DAEFRHDSGYEVHHQKLVFFAENVGSNKAI (SEQ ID NO. 57)

The variants of Aβ₁₋₃₀ of SEQ ID NO. 63 are shown by the following SEQ ID NO. 58.

(SEQ ID NO. 58)

X₂X₁X₂X₆X₄X₄X₂X₁₀X₁X₆X₂X₃X₄X₄X₉X₄X₃X₃X₆X₆X₁X₂X₂X₃
X₁X₅X₉X₄X₁X₃
or
X₁₂X₁X₂X₆X₄X₄X₂X₁₀X₁X₆X₂X₃X₄X₄X₉X₄X₃X₃X₆X₆X₁X₂X₂X₃
X₁X₅X₉X₄X₁X₃
X₁₂X₂X₁X₂X₆X₄X₄X₂X₁₀X₁X₆X₁₁X₄X₄X₉X₄X₁₁X₆X₆X₁X₁₁X₁
X₅X₉X₄X₁X₃
or
X₁₂X₂X₁X₂X₆X₄X₄X₂X₁₀X₁X₆X₁₁X₄X₄X₉X₄X₁₁X₆X₆X₁X₁3X₁
X₅X₉X₄X₁X₃

Where the peptides contain two different epitopes, the first epitope is at the aa location 1-11 or Aβ₁₋₁₁ as shown by the SEQ ID NO. 59.

(SEQ ID NO. 59)

DAEFRHDSGYE

Variants of SEQ ID NO. 59 are shown by the following SEQ ID NO. 60.

(SEQ ID NO. 60)

X₂X₁X₂X₆X₄X₄X₂X₁₀X₁X₆X₂
or
X₁₂X₂X₁X₂X₆X₄X₄X₂X₁₀X₁X₆X₂

The second epitope for Aβ₂₂₋₂₈ is at location 22-28 and is shown as SEQ ID NO. ENVGSNK (SEQ ID NO. 61) and the variants thereof are shown as SEQ ID NO. 62.

(SEQ ID NO. 62)

X₂X₂X₃X₁X₅X₉X₄
or
X₁₂X₂X₂X₃X₁X₅X₉X₄
or
X₁₂X₁₁X₁₁X₁X₅X₉X₄
or
X₁₂X₁3X₁X₅X₉X₄

An immunoreactive effect can also be elicited by absorption removal of a much smaller portion of the first 12 amino acids Aβ₁₋₁₂ shown as SEQ ID NO. 63 (Lemere et al., Nasal Aβ Treatment Induces Anti-Aβ Antibody Production and Decreases Cerebral Amyloid Burden in PD-APP Mice 2000 Ann. N.Y. Acad. Sci. 920:328).

(SEQ ID NO. 63)

DAEFRHDSGYEV

The variants of Aβ₁₋₁₂ are shown as SEQ ID NO. 64.

(SEQ ID NO. 64)

X₂X₁X₂X₆X₄X₄X₂X₁₀X₁X₆X₂X₃
or
X₁₂X₂X₁X₂X₆X₄X₄X₂X₁₀X₁X₆X₂X₃
or
X₁₂X₂X₁X₂X₆X₄X₄X₂X₁₀X₁X₆X₁₁

The first 10 amino acids can further be limited to the antigenic site as Aβ₁₋₁₀ shown as SEQ ID NO. 65.

(SEQ ID NO. 65)

DAEFRHDSGY

Variants of Aβ₁₋₁₀ are shown as SEQ ID NO. 66.

(SEQ ID NO. 66)

X₂X₁X₂X₆X₄X₄X₂X₁₀X₁X₆
or
X₁₂X₂X₁X₂X₆X₄X₄X₂X₁₀X₁X₆

Still further, a tetrapeptide of amino acids when incorporated into a phage epitope delivery system can be used to induce anti-aggregation activity antibodies for Aβ₃₋₆ as shown in SEQ ID NO. 67

(SEQ ID NO. 67)

EFRH

The variants of Aβ₃₋₆ are shown as SEQ ID NO. 68.

(SEQ ID NO. 68)

X₂X₇X₄X₄
or
X₁₂X₂X₇X₄X₄

However, known methods compositions contemplate other regions of amyloid peptides. For example, WO9748792A1 teaches amyloid peptides for a transgenic mouse model. Similarly, WO 98/03644A1 and U.S. Pat. No. 5,811,633 teach Alzheimer protein gene transgenic models. Moreover, immunological approaches for treating AD including peptides and delivery technologies including the VLP technology and tetrameric peptide Aβ₃₋₆ are known (Frenkel et al., 2001 Generation of auto-antibodies towards Alzheimer's disease vaccination, Vaccine 19:2615).

In particular, U.S. Pat. No. 4,666,829 teaches Aβ peptides including 1-28 and conjugation to KLH for induction of immune responses. Delivery devices are also known for peptides. L.E.A.P.S. technology for peptide delivery devices include uses to replace among other molecules KLH. Moreover, U.S. Pat. No. 5,652,342 and U.S. Pat. No. 6,096,315, the contents of which are incorporated herein by reference, teach examples and sequences for Central Nervous System (CNS) diseases such as MS and diseases caused by prions such as Creutzfeldt-Jakob disease (CJD) and Mad cow disease including Alzheimer's dementia. These sequences are contemplated by the invention and can be linked to any of the TCBL's described herein.

P1 for Dilated Cardiac Myopathy (DCM) or Experimental Autoimmune Myocarditis (EAM)

The P1 epitope can be selected from cardiac proteins such as those found reactogenic in Dilated Cardiac Myopathy (DCM) or Experimental Autoimmune Myocarditis (EAM) (Donermeyer et al., Myocarditis-inducing epitope of myosin binds constitutively and stably to I-Ak on antigen-presenting cells in the heart, J Exp Med 1995; 182:1291-1300; Bachmaier et al. 1999 “Generation of humanized mice susceptible to peptide induced inflammatory heart disease” Circulation 99:1885; Taylor et al., A spontaneous model for autoimmune myocarditis using the human MHC molecule HLA-DQ8. J Immunol. 2004 Feb. 15; 172(4):2651-8; Nishimura et al., “Autoimmune dilated cardiomyopathy in PD-1 receptor-deficient mice, Science, 2001 Jan. 12; 291(5502):319-22; Okazaki et al., “Autoantibodies against cardiac troponin I are responsible for dilated cardiomyopathy in PD-1-deficient mice”, Nat Med. 2003 December; 9(12):1477-83). A/J Mice Experimental Autoimmune Myocarditis (EAM) can be induced by infection with coxsackie virus B3 or by immunization with murine or porcine cardiac myosin or a cardiogenic peptide derived from the murine cardiac myosin. The My-1₃₃₄₋₃₅₂ is shown by the following SEQ ID NO. 16.

(SEQ ID NO. 16)

DSAFDVLSFTAEEKAGVYK

The improved variants of My-1₃₃₄₋₃₅₂ can be shown by the following SEQ ID NO. 69.

(SEQ ID NO. 69)

X₂X₅X₁X₆X₂X₃X₃X₅X₆X₁₀X₁X₂X₂X₄X₁X₁X₃X₆X₄
or
X₁₂X₂X₅X₁X₆X₂X₃X₃X₅X₆X₁₀X₁X₂X₂X₄X₁X₁X₃X₆X₄
or
X₁₂X₂X₅X₁X₆X₁₁X₅X₆X₁₀X₁X₁₁X₄X₁X₁X₃X₆X₄
or
X₁₂X₂X₅X₁X₆X₁3X₅X₆X₁₀X₁X₁₁X₄X₁X₁X₃X₆X₄

Another peptide that can be used from murine cardiac myosin is mM7α shown as SEQ ID NO. 70.

(SEQ ID NO. 70)

SLKLMATLFSTYAS

A variant for mM7α is shown as SEQ ID NO. 71.

(SEQ ID NO. 71)

X₅X₃X₄X₃X₈X₁X₁₀X₃X₇X₅X₁₀X₆X₁X₅

The corresponding human peptide hM7α is shown as SEQ ID NO. 72.

(SEQ ID NO. 72)

SLKLMATLFSSYAT

The variants of hM7α are shown as SEQ ID NO. 73.

(SEQ ID NO. 73)

X₅X₃X₄X₃X₈X₁X₁₀X₃X₇X₅X₅X₆X₁X₁₀
or
X₁₂X₅X₃X₄X₃X₈X₁X₁₀X₃X₇X₅X₅X₆X₁X₁₀

SEQ ID NO.'s 16 and 69-73 cause Experimental Autoimmune Myocarditis (EAM) in a line of double transgenic mice negative for both murine CD4 and CD8 containing human CD4 and DQ6 in the C57BL6 strain (Bachmaier et al. supra). Similarly, mM7α causes EAM in BALB/c but not the C57BL6, or the single transgenic hCD4 in a mCD4, mCD8 double knockout (Id.). Human DQ6 has been implicated in myocarditis in man (Limas et al., “Possible involvement of the HLA-DQB1 gene in susceptibility and resistance to human dilated cardiomyopathy”, Am Heart J. 1995 June; 129(6):1141-4).

Other human epitopes of myosin associated with induction of myocarditis in rats are encompassed by SEQ ID NO.'s 16 and 69-73. Still others are found in the S2 region (Li et al., Cryptic Epitope Identified in Rat and Human Cardiac Myosin S2 R Induces Myocarditis in the Lewis Rat, J Immunol. 2004. 172:3225-3234) and include S2-16, the sequence being shown by SEQ ID NO. 769.

(SEQ ID NO. 769)

KRKLEGDLKLTQESIMDLENDKQQL

The improved variants of the S2 region are shown as SEQ ID NO. 770.

(SEQ ID NO. 770)

X₄X₄X₄X₃X₂X₁X₂X₃X₄X₃X₁₀X₉X₃X₁₀X₃X₈X₂X₃X₂X₉X₂X₄X₉X₉
X₃
or
X₁₂X₄X₄X₄X₃X₂X₁X₂X₃X₄X₃X₁₀X₉X₃X₁₀X₃X₈X₂X₃X₂X₉X₂X₄
X₉X₉X₃
or
X₄X₄X₄X₁₁X₁X₁₁X₄X₃X₁₀X₉X₃X₁₀X₃X₈X₁₁X₂X₉X₂X₄X₉X₉X₃
or
X₁₂X₄X₄X₄X₁₁X₁X₁₁X₄X₃X₁₀X₉X₃X₁₀X₃X₈X₁₁X₂X₉X₂X₄X₉X₉
X₃
or
X₄X₄X₄X₃X₂X₁X₂X₃X₄X₃X₁₀X₉X₃X₁₀X₃X₈X₁3X₉X₂X₄X₉X₉X₃
or
X₁₂X₄X₄X₄X₃X₂X₁X₂X₃X₄X₃X₁₀X₉X₃X₁₀X₃X₈X₁3X₉X₂X₄X₉X₉
X₃

It is also contemplated that antibodies identified in a patients sera could be evaluated with regard to epitope suitability in a L.E.A.P.S. conjugate for therapeutic application (Anderson et al., Molecular analysis of polyreactive monoclonal antibodies from rheumatic carditis: human anti-N-acetylglucosamine/anti-myosin antibody V region genes, J Immunol. 1998 Aug. 15; 161(4):2020-31).

The murine and human sequences for cardiac antigen troponin I are the same wherein both sequences (have been implicated in human disease) called cTnIξ, the sequence being shown by SEQ ID NO. 74.

(SEQ ID NO. 74)

NITEIADLTQK

The improved variants of cTnξ as shown by the SEQ ID NO. 75 are

(SEQ ID NO. 75)

X₉X₃X₁₀X₂X₃X₁X₂X₃X₁₀X₉X₄
or
X₁₂X₉X₃X₁₀X₂X₃X₁X₂X₃X₁₀X₉X₄
or
X₁₂X₉X₃X₁₀X₁₁X₁X₁₁X₁₀X₉X₄

It is also known that a knockout mouse line having the gene PD-1 for programmed cell death 1 being deleted develop Dilated Cardiomyopathy (DCM) with anti cardiac tropinin (cTnI) antibodies. Some of the antibodies are capable of recognizing a single peptide epitope and the monoclonal antibodies from cloned lines from these mice are capable of generating some of the EAM effects seen in the transgenic mice (Okazaki et al., supra). Other transgenic mice have been prepared using human genes implicated in myocarditis such as HLA DQ8 (Taylor et al., supra; Elliott et al., Autoimmune cardiomyopathy and heart block develop spontaneously in HLA-DQ8 transgenic IAbeta knockout NOD mice, Proc Natl Acad Sci USA 2003 Nov. 11, 100(23):13447-52). Transgenic mice have also been prepared using human cardiac protein genes such as myotrophin (Sarkar et al., “Cardiac over-expression of Myotrophin triggers myocardial hypertrophy and heart failure in transgenic mice”, JBC epub Feb. 16, 2004, J Biol Chem. 2004 Feb. 16).

Any of these sequences isolated from murine knockouts or isolated from the antibodies found therein are contemplated by the invention and can be linked to any of the TCBL's described herein.

Ansari et al. (1994 “Epitope mapping of the branched chain alpha-ketoacid dehydrogenase dihydrolipoyl transacylase (BCKD-E2) protein that reacts with sera from patients with idiopathic dilated cardiomyopathy”, J Immunol 153(10): 4754-65) identified the peptide BCKD-E2₁₁₆₋₁₃₄, the sequence being shown by SEQ ID NO. 771.

(SEQ ID NO. 771)

VRRLAMENNIKLSEVVGSG

The improved variants of BCKD-E2₁₁₆₋₁₃₄ are shown by SEQ ID NO. 772.

(SEQ ID NO. 772)

X₃X₄X₄X₃X₁X₈X₂X₉X₉X₃X₄X₃X₁₀X₂X₃X₃X₁X₁₀X₁
or
X₁₂X₃X₄X₄X₃X₁X₈X₂X₉X₉X₃X₄X₃X₁₀X₂X₃X₃X₁X₁₀X₁
or
X₃X₄X₄X₃X₁X₈X₂X₉X₉X₃X₄X₃X₁₀X₁₁X₃X₁X₁₀X₁
or
X₃X₄X₄X₃X₁X₈X₂X₉X₉X₃X₄X₃X₁₀X₁3X₁X₁₀X₁
or
X₁₂X₃X₄X₄X₃X₁X₈X₂X₉X₉X₃X₄X₃X₁₀X₁₁X₃X₁X₁₀X₁
or
X₁₂X₃X₄X₄X₃X₁X₈X₂X₉X₉X₃X₄X₃X₁₀X₁3X₁X₁₀X₁

Adderson et al. (1998. Molecular analysis of polyreactive monoclonal antibodies from rheumatic carditis: human anti-N-acetylglucosamine/anti-myosin antibody V region genes. J Immunol. 161:2020-31) identified the LMM1 peptide as follows as shown by SEQ ID NO. 773.

(SEQ ID NO. 773)

KEALISSLTRGKLTYTQQ

The improved variants of the LLM1 peptide are shown by SEQ ID NO. 774.

(SEQ ID NO. 774)

X₄X₂X₁X₃X₃X₁₀X₁₀X₃X₁₀X₄X₁X₄X₃X₁₀X₆X₁₀X₉X₉
or
X₁₂X₄X₂X₁X₃X₃X₁₀X₁₀X₃X₁₀X₄X₁X₄X₃X₁₀X₆X₁₀X₉X₉
or
X₄X₂X₁X₁₁X₁₀X₁₀X₃X₁₀X₄X₁X₄X₃X₁₀X₆X₁₀X₉X₉
or
X₁₂X₄X₂X₁X₁₁X₁₀X₁₀X₃X₁₀X₄X₁X₄X₃X₁₀X₆X₁₀X₉X₉

Adderson et al. also identified the LMM33 peptide as shown by SEQ ID NO. 775.

SERVQLLHSQNTSLINQK (SEQ ID NO. 775)

The improved variants the LMM33 peptide as shown by SEQ ID NO. 776.

(SEQ ID NO. 776)

X₁₀X₂X₄X₃X₉X₃X₃X₄X₁₀X₉X₉X₁₀X₁₀X₃X₃X₁₀X₁₀X₄
or
X₁₀X₂X₄X₃X₉X₃X₃X₄X₁₀X₉X₉X₁₀X₁₀X₃X₃X₁₀X₁₀X₄
or
X₁₂X₁₀X₂X₄X₃X₉X₃X₃X₄X₁₀X₉X₉X₁₀X₁₀X₃X₃X₁₀X₁₀X₄
or
X₁₂X₁₀X₂X₄X₃X₉X₁₁X₄X₁₀X₉X₉X₁₀X₁₀X₁₁X₁₀X₁₀X₄

P1 for Peptide Antigen for Insulin Dependent Diabetes Mellitus (IDDM)

It has been known in the art that an islet cell 65 kDa protein and peptides from bovine serum albumin (BSA), in particular one called ABBOS, an enzyme Glutamic Acid Decarboxylase (GAD65) found in pancreatic beta islet and other cells including brain, CVB4, Heat Shock Protein (HSP) and insulin, Bacterial Superantigens (BSA), CVB4 and rubella virus have been implicated as environmental factors causing Insulin Dependent Diabetes Mellitus (IDDM) in a susceptible host, perhaps by molecular mimicry and epitope spreading to encompass a natural epitope of GAD or HSP. The peptides that have been suggested as causative antigens of IDDM can be linked to the TCBL ligands described herein to form a therapeutic for the treatment of IDDM.

The abbreviation for the amino acid sequence is GAD65 wherein the sequence starting at position is 247 is described by SEQ ID NO. 76 (Wilson et al., Therapeutic Alteration of Insulin-Dependent Diabetes Mellitus Progression by T Cell Tolerance to Glutamic Acid Decarboxylase 65 Peptides In Vitro and In Vivo, 2001, J. Immunol., 167: 569, Tisch et al., supra 1999 JI 163:1178; Karlsson et al., Th1-like dominance in high-risk first-degree relatives of type I diabetic patients 2000, Diabetologia 43:742).

(SEQ ID NO. 76)

NMYAMMIARFKMFPEVKEKGMAALPRLIAFTSEHSHFSLK

The improved variants of the GAD65 starting at position is 247 are

(SEQ ID NO. 77)

X₉X₈X₆X₁X₈X₈X₃X₁X₄X₆X₄X₈X₆X₇X₂X₃X₄X₂X₄X₁X₈X₁X₁X₃X₇
X₄X₃X₃X₁X₆X₁₀X₅X₂X₄X₅X₄X₆X₅X₃X₄
or
X₁₂X₉X₈X₆X₁X₈X₈X₃X₁X₄X₆X₄X₈X₆X₇X₂X₃X₄X₂X₄X₁X₈X₁X₁
X₃X₇X₄X₃X₃X₁X₆X₁₀X₅X₂X₄X₅X₄X₆X₅X₃X₄
or
X₁₂X₉X₈X₆X₁X₈X₈X₃X₁X₄X₆X₄X₈X₆X₇X₁₁X₄X₂X₄X₁X₈X₁X₁X₃
X₇X₄X₁₁X₁X₆X₁₀X₅X₂X₄X₅X₄X₆X₅X₃X₄

Another GAD65 peptide starting at position 253 is shown by SEQ ID NO. 78.

(SEQ ID NO. 78)

IARFKMFPEVKEKGMAALPRLIAFTSEHSHFSLK

The improved variants of the GAD65 peptide starting at position 253 are shown by SEQ ID NO. 79.

(SEQ ID NO. 79)

X₃X₁X₄X₆X₄X₈X₆X₇X₂X₃X₄X₂X₄X₁X₈X₁X₁X₃X₇X₄X₃X₃X₁X₆
X₁₀X₅X₂X₄X₅X₄X₆X₅X₃X₄
or
X₁₂X₃X₁X₄X₆X₄X₈X₆X₇X₂X₃X₄X₂X₄X₁X₈X₁X₁X₃X₇X₄X₃X₃X₁
X₆X₁₀X₅X₂X₄X₅X₄X₆X₅X₃X₄
or
X₁₂X₃X₁X₄X₆X₄X₈X₆X₇X₂X₃X₄X₂X₄X₁X₈X₁X₁X₃X₇X₄X₁₁X₁X₆
X₁₀X₅X₂X₄X₅X₄X₆X₅X₃

The GAD65 starting at position 524 is shown by SEQ ID NO. 80 (Quinn et al., MHC Class I-Restricted Determinants on the Glutamic Acid Decarboxylase 65 Molecule Induce Spontaneous CTL Activity, 2001, J. Immunol., 167:1748, Herman A et al., Determination of Glutamic Acid Decarboxylase 65 Peptides Presented by the Type I Diabetes-Associated HLA-DQ8 Class II Molecule Identifies an Immunogenic Peptide Motif 1999 JI 163:6275; Winer et al., Peptide Dose, MHC Affinity, and Target Self-Antigen Expression Are Critical for Effective Immunotherapy of Nonobese Diabetic Mouse Prediabetes 2000 JI 165:4086).

SRLSKVAPVIKARMMEYGTT (SEQ ID NO. 80)

The improved variants of the GAD65 peptide starting at position 524 are shown by SEQ ID NO. 81.

(SEQ ID NO.81)

X₅X₄X₃X₅X₄X₃X₁X₇X₃X₃X₄X₁X₄X₈X₈X₂X₆X₁X₁₀X₁₀
or
X₁₂X₅X₄X₃X₅X₄X₃X₁X₇X₃X₃X₄X₁X₄X₈X₈X₂X₆X₁X₁₀X₁₀
or
X₁₂X₅X₄X₃X₅X₄X₃X₁X₇X₁₁X₄X₁X₄X₈X₈X₂X₆X₁X₁₀X₁₀

The GAD65 starting at position 506 is shown by SEQ ID NO. 82 (Wilson et al., supra Karlsson et al., supra).

(SEQ ID NO. NO. 82)

IPPSLRYLEDEERMSRLSK

The improved variants of the GAD65 peptide starting at position 506 are shown by SEQ ID NO. 83.

(SEQ ID NO. 83)

X₃X₇X₇X₅X₃X₄X₆X₂X₂X₂X₂X₄X₈X₅X₄X₃X₅X₄
or
X₁₂X₃X₇X₇X₅X₃X₄X₆X₂X₂X₂X₂X₄X₈X₅X₄X₃X₅X₄
or
X₁₂X₃X₇X₇X₅X₃X₄X₆X₁₁X₁₁X₄X₈X₅X₄X₃X₅X₄
or
X₁₂X₃X₇X₇X₅X₃X₄X₆X₁3X₄X₈X₅X₄X₃X₅X₄

The GAD65 starting at position 201 is shown by the SEQ ID NO. 84 (Quinn et al., supra JI 167:1748 2001, Herman A et al., supra 1999 JI 163:6275, Liu J, et al., Major DQ8-restricted T-cell epitopes for human GAD65 mapped using human CD4, DQA1*0301, DQB1*0302 transgenic IA (null) NOD mice Diabetes. 1999 March; 48(3):469-77).

(SEQ ID NO. 84)

NTNMFTYEIAPVFVLLEYVT

The improved variants of the GAD65 peptide starting at position 201 are shown by SEQ ID NO. 85.

(SEQ ID NO. 85)

X₉X₁₀X₉X₈X₆X₁₀X₆X₂X₃X₁X₇X₃X₆X₃X₃X₃X₂X₆X₃X₁₀
or
X₁₂X₉X₁₀X₉X₈X₆X₁₀X₆X₂X₃X₁X₇X₃X₆X₃X₃X₃X₂X₆X₃X₁₀
or
X₁₂X₉X₁₀X₉X₈X₆X₁₀X₆X₁₁X₁X₇X₃X₆X₁₁X₁₁X₆X₃X₁₀
or
X₁₂X₉X₁₀X₉X₈X₆X₁₀X₆X₁₁X₁X₇X₃X₆X₁3X₆X₃X₁₀

The peptide from Bovine Serum albumin called ABBOS at position 152 is shown by the SEQ ID NO. 86 (Winer et al., supra 2000 JI 165:4086, Karlsson et al., supra Diabetologia 43:742).

(SEQ ID NO. 86)

FKADEKKFWGKYLYE

The improved variants of ABBOS at position 152 are shown by SEQ ID NO. 87.

(SEQ ID NO. 87)

X₆X₄X₁X₂X₂X₄X₄X₆X₆X₁X₄X₆X₃X₆X₂
or
X₁₂X₆X₄X₁X₂X₂X₄X₄X₆X₆X₁X₄X₆X₃X₆X₂
or
X₁₂X₆X₄X₁X₁₁X₄X₄X₆X₆X₁X₄X₆X₃X₆X₂

The human Insulin β chain position starting at aa9 is shown by SEQ ID NO. 88 (Urbank-Ruiz et al., Immunization with DNA encoding an immunodominant peptide of insulin prevents diabetes in NOD mice, 2001, Clin Immunol 100 (2): 164-171, Abiru et al., Peptide and Major Histocompatibility Complex-Specific Breaking of Humoral Tolerance to Native Insulin With the B9-23 Peptide in Diabetes-Prone and Normal Mice Diabetes 50:1274 2001).

(SEQ ID NO. 88)

SHLVEALYLVCGERG

The improved variants human Insulin β chain position starting at aa9 are shown by SEQ ID NO. 89.

(SEQ ID NO. 89)

X₅X₄X₃X₃X₂X₁X₃X₆X₃X₅X₁X₂X₄X₁
or
X₁₂X₅X₄X₃X₃X₂X₁X₃X₆X₃X₅X₁X₂X₄X₁
or
X₁₂X₅X₄X₁₁X₁X₃X₆X₃X₅X₁X₂X₄X₁
or
X₁₂X₅X₄X₁₃X₁X₃X₆X₃X₅X₁X₂X₄X₁

The human heat shock protein HSP at position 277 is shown by SEQ ID NO. 90 (Elias et al., Induction of diabetes in standard mice by immunization with the p277 peptide of a 60-kDa heat shock protein, 1995, Eur J Immunol 25:2815).

(SEQ ID NO. 90)

VLGGGCALLRCIPALDSLTPANED

The improved variants of the human heat shock protein HSP at position 277 are shown by SEQ ID NO. 91.

(SEQ ID NO. 91)

X₃X₃X₁X₁X₁X₅X₁X₃X₃X₄X₅X₃X₇X₁X₃X₂X₅X₃X₁₀X₇X₁X₉X₂X₂
or
X₁₂X₃X₃X₁X₁X₁X₅X₁X₃X₃X₄X₅X₃X₇X₁X₃X₂X₅X₃X₁₀X₇X₁X₉
X₂X₂
or
X₁₂X₁₁X₁X₁X₁X₅X₁X₁₁X₄X₅X₃X₇X₁X₁₁X₅X₃X₁₀X₇X₁X₉X₁₁

The RVEp151 is shown by SEQ ID NO. 92 (Ou et al., Cross-reactive rubella virus and glutamic acid decarboxylase (65 and 67) protein determinants recognised by T cells of patients with type I diabetes mellitus, 2000, Diabetologia 43:750).

(SEQ ID NO. 92)

EACVTSWLWSGEGAVFYRVDLHFINLGT

The improved variants of RVEp151 are shown by SEQ ID NO. 93.

(SEQ ID NO. 93)

X₂X₁X₅X₃X₁₀X₅X₆X₃X₆X₅X₁X₂X₁X₁X₃X₆X₆X₄X₃X₂X₃X₄X₆X₃
X₉X₃X₁X₁₀
or
X₁₂X₂X₁X₅X₃X₁₀X₅X₆X₃X₆X₅X₁X₂X₁X₁X₃X₆X₆X₄X₃X₂X₃X₄X₆
X₃X₉X₃X₁X₁₀
or
X₁₂X₂X₁X₅X₃X₁₀X₅X₆X₃X₆X₅X₁X₂X₁X₁X₃X₆X₆X₄X₁₁X₄X₆X₃
X₉X₃X₁X₁₀
or
X₁₂X₂X₁X₅X₃X₁₀X₅X₆X₃X₆X₅X₁X₂X₁X₁X₃X₆X₆X₄X₁₃X₄X₆X₃
X₉X₃X₁X₁₀

The RVEp87 is shown by SEQ ID NO. 94 (Ou et al., Diabetologia 43:750 2000).

(SEQ ID NO. 94)

MDFWCVEHDRPPPATPTSLTT

The improved variants of RVE p87 are shown by SEQ ID NO. 95.

(SEQ ID NO. 95)

X₈X₂X₆X₆X₅X₃X₂X₄X₂X₄X₇X₇X₇X₁X₁₀X₇X₁₀X₅X₃X₁₀X₁₀
or
X₁₂X₈X₂X₆X₆X₅X₃X₂X₄X₂X₄X₇X₇X₇X₁X₁₀X₇X₁₀X₅X₃X₁₀X₁₀
or
X₁₂X₈X₂X₆X₆X₅X₁₁X₄X₂X₄X₇X₇X₇X₁X₁₀X₇X₁₀X₅X₃X₁₀X₁₀

The GAD65 from positions 247 to 265 is shown by SEQ ID NO. 96.

(SEQ ID NO. 96)

NMYAMMIARFKMFPEVKEKG

The improved variants GAD65 from positions 247 to 265 are shown by SEQ ID NO. 97.

(SEQ ID NO. 97)

X₉X₈X₆X₁X₈X₈X₃X₁X₄X₆X₄X₈X₆X₇X₂X₃X₄X₂X₄X₁
or
X₁₂X₉X₈X₆X₁X₈X₈X₃X₁X₄X₆X₄X₈X₆X₇X₂X₃X₄X₂X₄X₁
or
X₁₂X₉X₈X₆X₁X₈X₈X₃X₁X₄X₆X₄X₈X₆X₇X₁₁X₄X₂X₄X₁

The GAD65 from positions 247 to 279 is shown as SEQ ID NO. 98 (Wilson et al., supra JI 167:569 2001 Tisch et al., supra 1999 JI 163: 1178).

(SEQ ID NO. 98)

NMYAMMIARFKMFPEVKEKGMAALPRLIAFTSE

The improved variants of GAD65 from positions 247 to 279 are shown by SEQ ID NO. 99.

(SEQ ID NO. 99)

X₉X₈X₆X₁X₈X₈X₃X₁X₄X₆X₄X₈X₆X₇X₂X₃X₄X₂X₄X₁X₈X₁X₁X₃X₇
X₄X₃X₃X₁X₆X₁₀X₅X₂
or
X₁₂X₉X₈X₆X₁X₈X₈X₃X₁X₄X₆X₄X₈X₆X₇X₂X₃X₄X₂X₄X₁X₈X₁X₁
X₃X₇X₄X₃X₃X₁X₆X₁₀X₅X₂
or
X₁₂X₉X₈X₆X₁X₈X₈X₃X₁X₄X₆X₄X₈X₆X₇X₁₁X₄X₂X₄X₁X₈X₁X₁X₃
X₇X₄X₁₁X₁X₆X₁₀X₅X₂

The GAD65 at position 274 is shown as SEQ ID NO. 100 (Karlsson et al., supra, Diabetologia 43:742).

(SEQ ID NO. 100)

IAFTSEHSHFSLK

The improved variants of GAD65 at position 274 are shown by SEQ ID NO. 101.

(SEQ ID NO. 101)

X₃X₁X₆X₁₀X₅X₂X₄X₅X₄X₆X₅X₃X₄
or
X₁₂X₃X₁X₆X₁₀X₅X₂X₄X₅X₄X₆X₅X₃X₄

The RVE at position 87 is shown as SEQ ID NO. 102 (Quinn et al., supra JI 167:1748 2001, Herman et al., supra 1999 JI 163:6275, Liu et al., supra Diabetes. 1999 March; 48(3):469-77; Urbank-Ruiz et al., supra Clin Immunol 100 (2): 164-171 2001; Abiru et al., supra Diabetes 50:1274 2001; Elias et al., supra Eur J Immunol 25:2815 1995; Ou et al., supra Diabetologia 43:750 2000).

(SEQ ID NO. 102)

MDFWCVEHDRPPPATPTSLTT

The improved variants of RVE at position 87 are shown by SEQ ID NO. 103.

(SEQ ID NO. 103)

X₈X₂X₆X₆X₅X₃X₂X₄X₂X₄X₇X₇X₇X₁X₁₀X₇X₁₀X₅X₃X₁₀X₁₀
or
X₁₂X₈X₂X₆X₆X₅X₃X₂X₄X₂X₄X₇X₇X₇X₁X₁₀X₇X₁₀X₅X₃X₁₀X₁₀
or
X₁₂X₈X₂X₆X₆X₅X₁₁X₄X₂X₄X₇X₇X₇X₁X₁₀X₇X₁₀X₅X₃X₁₀X₁₀

The GAD65 at positions 104-112 is shown by SEQ ID NO. 104 (Ou et al., supra; Sadeharju et al., Enterovirus antibody levels during the first two years of life in prediabetic autoantibody-positive children, 2001 Diabetologia 44(7):818).

(SEQ ID NO. 104)

ERPTLAFLQD

The improved variants of GAD65 at positions 104-112 are shown by SEQ ID NO. 105.

(SEQ ID NO. 105)

X₂X₄X₇X₁₀X₃X₁X₆X₃X₉X₂
or
X₁₂X₂X₄X₇X₁₀X₃X₁X₆X₃X₉X₂

The GAD65 at positions 127-136 is shown by SEQ ID NO. 106 (Herman et al., supra).

(SEQ ID NO. 106)

DRSTKVIDFH

The improved variants of GAD65 at positions 127-136 are shown by SEQ ID NO. 107.

(SEQ ID NO. 107)

X₂X₄X₅X₁₀X₄X₃X₃X₂X₆X₄
or
X₁₂X₂X₄X₅X₁₀X₄X₃X₃X₂X₆X₄
or
X₁₂X₂X₄X₅X₁₀X₄X₁₁X₆X₄
or
X₁₂X₂X₄X₅X₁₀X₄X₁₃X₆X₄

Peptides from a protein referred to as Type 1 DM-associated autoantigen insulinoma-associated-2 are also contemplated by the invention (Peakman 1999, “Naturally processed and presented epitopes of the islet cell autoantigen IA-2 eluted from HLA-DR4”, J. Clin Invest 04:1449-1457).

The peptide at positions 654-674 is shown by the SEQ ID NO. 461.

(SEQ ID NO. 461)

VSSVSSQFSDAAQASPSSHSS

The peptide improved variants are as follows.

(SEQ ID NO. 462)

X₃X₁₀X₁₀X₃X₁₀X₁₀X₉X₇X₁₀X₂X₁X₁X₁₀X₁X₁₀X₇X₁₀X₁₀X₄X₁₀
X₁₀
or

(SEQ ID NO. 464)

X₁₂X₃X₁₀X₁₀X₃X₁₀X₁₀X₉X₇X₁₀X₂X₁X₁X₁₀X₁X₁₀X₇X₁₀X₁₀X₄
X₁₀X₁₀

The SEQ ID NO. 465 shows the peptide at positions 797-817 (Peakman Id.).

(SEQ ID NO. 465)

MVWESGCTVIVMLTPLVEDGV

The improved variants of the SEQ ID NO. 465 are as follows.

(SEQ ID NO. 466)

X₈X₃X₆X₂X₁₀X₁X₅X₁₀X₃X₃X₃X₈X₃X₃X₇X₃X₃X₂X₂X₁X₃
or

(SEQ ID NO. 467)

X₁₂X₈X₃X₆X₂X₁₀X₁X₅X₁₀X₃X₃X₃X₈X₃X₃X₇X₃X₃X₂X₂X₁X₃
or

(SEQ ID NO. 468)

X₈X₃X₆X₂X₁₀X₁X₅X₁₀X₁₁X₃X₈X₃X₃X₇X₃X₃X₂X₂X₁X₃
or

(SEQ ID NO. 469)

X₈X₃X₆X₂X₁₀X₁X₅X₁₀X₃X₁₁X₈X₃X₃X₇X₃X₃X₂X₂X₁X₃
or

(SEQ ID NO. 470)

X₈X₃X₆X₂X₁₀X₁X₅X₁₀X₃X₃X₃X₈X₃X₃X₇X₃X₃X₁₁X₁X₃
or

(SEQ ID NO. 471)

X₈X₃X₆X₂X₁₀X₁X₅X₁₀X₁₃X₈X₃X₃X₇X₃X₃X₂X₂X₁X₃
or

(SEQ ID NO. 472)

X₈X₃X₆X₂X₁₀X₁X₅X₁₀X₃X₃X₃X₈X₃X₃X₇X₁₃X₁X₃
or

(SEQ ID NO. 473)

X₈X₃X₆X₂X₁₀X₁X₅X₁₀X₁₃X₈X₃X₃X₇X₁₃X₁X₃
or

(SEQ ID NO. 474)

X₈X₃X₆X₂X₁₀X₁X₅X₁₀X₁₁X₃X₈X₁₁X₇X₁₁X₁₁X₁X₃
or

(SEQ ID NO. 475)

X₁₂X₈X₃X₆X₂X₁₀X₁X₅X₁₀X₁₁X₃X₈X₃X₃X₇X₃X₃X₂X₂X₁X₃
or

(SEQ ID NO. 476)

X₁₂X₈X₃X₆X₂X₁₀X₁X₅X₁₀X₃X₁₁X₈X₃X₃X₇X₃X₃X₂X₂X₁X₃
or

(SEQ ID NO. 477)

X₁₂X₈X₃X₆X₂X₁₀X₁X₅X₁₀X₃X₃X₃X₈X₃X₃X₇X₃X₃X₁₁X₁X₃
or

(SEQ ID NO. 478)

X₁₂X₈X₃X₆X₂X₁₀X₁X₅X₁₀X₁₃X₈X₃X₃X₇X₃X₃X₂X₂X₁X₃
or

(SEQ ID NO. 479)

X₁₂X₈X₃X₆X₂X₁₀X₁X₅X₁₀X₃X₃X₃X₈X₃X₃X₇X₁₃X₁X₃
or

(SEQ ID NO. 480)

X₁₂X₈X₃X₆X₂X₁₀X₁X₅X₁₀X₁₃X₈X₃X₃X₇X₁₃X₁X₃
or

(SEQ ID NO. 481)

X₁₂X₈X₃X₆X₂X₁₀X₁X₅X₁₀X₁₁X₃X₈X₁₁X₇X₁₁X₁₁X₁X₃

The SEQ ID NO. 482 shows the peptide at positions 854-872 (Peakman Id.).

(SEQ ID NO. 482)

FYLKNVQTQETRTLTQFHF

The improved variants of as follows SEQ ID NO. 482 are as follows.

(SEQ ID NO. 483)

X₇X₆X₃X₄X₉X₃X₉X₁₀X₉X₂X₃X₄X₁₀X₃X₁₀X₉X₇X₄X₆
or

(SEQ ID NO. 484)

X₁₂X₇X₆X₃X₄X₉X₃X₉X₁₀X₉X₂X₃X₄X₁₀X₃X₁₀X₉X₇X₄X₆
or

(SEQ ID NO. 485)

X₇X₆X₃X₄X₉X₃X₉X₁₀X₉X₁₁X₄X₁₀X₃X₁₀X₉X₇X₄X₆
or

(SEQ ID NO. 486)

X₁₂X₇X₆X₃X₄X₉X₃X₉X₁₀X₉X₁₁X₄X₁₀X₃X₁₀X₉X₇X₄X₆

P1 for Rheumatoid Arthritis

Various antigens often with defined epitopes recognized for rheumatoid arthritis (RA) are collagen type II 390-402.

For example, the collagen type II 390-402 as previously described as SEQ ID NO. 1 is shown below.

(SEQ ID NO. 1)

IAGFKGEQGPKGE

The improved variants of collagen type II 390-402 are as follows.

(SEQ ID NO. 108)

X₃X₁X₁X₆X₄X₁X₂X₉X₁X₇X₄X₁X₂
or
X₁₂X₃X₁X₁X₆X₄X₁X₂X₉X₁X₇X₄X₁X₂

Peptides from Type II human collagen (Krco et al. (1999 Identification of T cell determinants on human type II collagen recognized by HLA-DQ8 and HLA-DQ6 transgenic mice. J Immunol.163(3):1661-5) referred therein as Peptide G 54-73 is shown by the SEQ ID NO. 487.

(SEQ ID NO. 487)

DGEAGKPGKAGERGPPGPQG

The improved variants from Type II human collagen are as follows.

(SEQ ID NO. 488)

X₂X₁X₂X₁X₁X₄X₇X₁X₄X₁X₁X₂X₄X₁X₇X₇X₁X₇X₉X₁
or

(SEQ ID NO. 489)

X₁₂X₂X₁X₂X₁X₁X₄X₇X₁X₄X₁X₁X₂X₄X₁X₇X₇X₁X₇X₉X₁

Peptide K at positions 94-113 is shown by SEQ ID NO. 490 (Krco et al., id.).

(SEQ ID NO. 490)

GLDGAKGEAGAPGVKGESGS

The improved variants of Peptide K at positions 94-113 SEQ ID NO. 490 are shown as follows.

(SEQ ID NO. 491)

X₁X₃X₂X₁X₁X₄X₁X₂X₁X₁X₁X₇X₁X₃X₄X₁X₂X₁₀X₁X₁₀
or

(SEQ ID NO. 492)

X₁₂X₁X₃X₂X₁X₁X₄X₁X₂X₁X₁X₁X₇X₁X₃X₄X₁X₂X₁₀X₁X₁₀
or

(SEQ ID NO. 493)

X₁X₁₁X₁X₁X₄X₁X₂X₁X₁X₁X₇X₁X₃X₄X₁X₂X₁₀X₁X₁₀
or

(SEQ ID NO. 494)

X₁₂X₁X₁₁X₁X₁X₄X₁X₂X₁X₁X₁X₇X₁X₃X₄X₁X₂X₁₀X₁X₁₀

Peptide 44 at positions 554-573 is shown by SEQ ID NO. 495 (Krco et al., id.).

(SEQ ID NO. 495)

FERGAAGIAGDKGDRGDVGEK

The improved variants of Peptide 44 at positions 554-573 of SEQ ID NO. 495 are shown as follows.

(SEQ ID NO. 496)

X₂X₄X₁X₁X₁X₁X₃X₁X₁X₂X₄X₁X₂X₄X₁X₂X₃X₁X₂X₄
or

(SEQ ID NO. 497)

X₁₂X₂X₄X₁X₁X₁X₁X₃X₁X₁X₂X₄X₁X₂X₄X₁X₂X₃X₁X₂X₄
or

(SEQ ID NO. 498)

X₂X₄X₁X₁X₁X₁X₃X₁X₁X₂X₄X₁X₂X₄X₁X₁₁X₁X₂X₄
or

(SEQ ID NO. 499)

X₁₂X₂X₄X₁X₁X₁X₁X₃X₁X₁X₂X₄X₁X₂X₄X₁X₁₁X₁X₂X₄

The protein Osteopontin (OPN) contains a peptide referred shown by the SEQ ID NO. 500 (Yamamoto et al., (2003) Essential role of the cryptic epitope SLAYGLR within osteopontin in a murine model of rheumatoid arthritis. J. Clin. Invest. 112:181-188).

(SEQ ID NO. 500)

SLAYGLR

The improved variants Osteopontin (OPN) are shown as follows.

(SEQ ID NO. 501)

X₁₀X₃X₁X₆X₁X₃X₄
or

(SEQ ID NO. 502)

X₁₂X₁₀X₃X₁X₆X₁X₃X₄

A peptide naJP1 is indicated by SEQ ID NO. 503 (Prakken et al. (2004), Epitope-specific immunotherapy induces immune deviation of proinflammatory T cells in rheumatoid arthritis, Proc Nat Acad Sci USA 101:4228-4233).

(SEQ ID NO. 503)

QKRAAYKQYGHAAFE

The improved variants of naJP1 are indicated by the following.

(SEQ ID NO. 504)

X₉X₄X₄X₁X₁X₆X₄X₉X₆X₁X₄X₁X₁X₇X₂
or

(SEQ ID NO. 505)

X₁₂X₉X₄X₄X₁X₁X₆X₄X₉X₆X₁X₄X₁X₁X₇X₂

A peptide dnaJPv is indicated by SEQ ID NO. 506 (Prakken et al., id.).

(SEQ ID NO. 506)

ERAAYDQYGHAAFE

The improved variants of dnaJPv are indicated by the following.

(SEQ ID NO. 507)

X₂X₄X₁X₁X₆X₂X₉X₆X₁X₄X₁X₁X₇X₂
or

(SEQ ID NO. 508)

X₁₂X₂X₄X₁X₁X₆X₂X₉X₆X₁X₄X₁X₁X₇X₂

P1 for Pemphigus Vulgaris (PV)

Epidermal cell adhesion molecule desmoglein epitopes of Pemphigus Vulgaris (PV) have been identified (Veldman et al. (2004), “T cell recognition of desmoglein 3 peptides in patients with Pemphigus Vulgaris and healthy individuals. J Immunol. 172:3883-92). The epitopes of interest are indicated as 7 Des3 peptides and in particular, DG3 (342-358) and DG3 (376-392). The epitopes are recognized exclusively by T cell clones from PP individuals.

For example, the DG3₃₄₂₋₃₅₈ epitope at positions at 342-358 is shown by SEQ ID NO. 509.

(SEQ ID NO. 509)

SVKLSIAVKNKAEFHQS

The improved versions of the DG3₃₄₂₋₃₅₈ epitope are shown by the following.

(SEQ ID NO. 510)

X₁₀X₃X₄X₃X₁₀X₃X₁X₃X₄X₉X₄X₁X₂X₇X₄X₉X₁₀
or

(SEQ ID NO. 511)

X₁₂X₁₀X₃X₄X₃X₁₀X₃X₁X₃X₄X₉X₄X₁X₂X₇X₄X₉X₁₀

The epitope DG3₃₇₆₋₃₉₂ at positions at 376-392 is shown by SEQ ID NO. 512.

(SEQ ID NO. 512)

NVREGIAFRPASKTFTV

The improved versions of the DG3₃₇₆₋₃₉₂ epitope are shown by the following.

(SEQ ID NO. 513)

X₉X₃X₄X₂X₁X₃X₁X₇X₄X₇X₁X₁₀X₄X₁₀X₇X₁₀X₃
or

(SEQ ID NO. 514)

X₁₂X₉X₃X₄X₂X₁X₃X₁X₇X₄X₇X₁X₁₀X₄X₁₀X₇X₁₀X₃

The epitope for the Dsg1 amino terminus of 45 kDa is shown by SEQ ID NO. 515 (Abreu-Velez et al. (2003), The tryptic cleavage product of the mature form of the bovine desmoglein 1 ectodomain is one of the antigen moieties immunoprecipitated by all sera from symptomatic patients affected by a new variant of endemic pemphigus. Eur J Dermatol. 13:359-66).

(SEQ ID NO. 515)

EXIKFAAAXREGED

The improved versions of the Dsg1 amino terminus epitope are shown by the following.

(SEQ ID NO. 516)

X₂XX₃X₄X₇X₁X₁X₁XX₄X₂X₁X₂X₂
or

(SEQ ID NO. 517)

X₁₂X₂XX₃X₄X₇X₁X₁X₁XX₄X₂X₁X₂X₂
or

(SEQ ID NO. 518)

X₁₁X₄X₇X₁X₁X₁XX₄X₂X₁X₂X₂
or

(SEQ ID NO. 519)

X₁₃X₄X₇X₁X₁X₁XX₄X₂X₁X₂X₂
or

(SEQ ID NO. 520)

X₁₁X₄X₇X₁X₁X₁XX₄X₂X₁X₁₁
or

(SEQ ID NO. 521)

X₁₃X₄X₇X₁X₁X₁XX₄X₂X₁X₁₁
or

(SEQ ID NO. 522)

X₁₂X₁₁X₄X₇X₁X₁X₁XX₄X₂X₁X₂X₂
or

(SEQ ID NO. 523)

X₁₂X₁₃X₄X₇X₁X₁X₁XX₄X₂X₁X₂X₂
or

(SEQ ID NO. 524)

X₁₂X₁₁X₄X₇X₁X₁X₁XX₄X₂X₁X₁₁
or

(SEQ ID NO. 525)

X₁₂X₁₃X₄X₇X₁X₁X₁XX₄X₂X₁X₁₁

P1 for Multiple Sclerosis

For Multiple Sclerosis (MS) P1 can be selected from among molecules such as Myelinproteolipid MPL which has the peptide sequence as shown by SEQ ID NO. 2 as previously described.

(SEQ ID NO. 2)

KNIVTPRT

The improved variants of the MPL peptide are shown by SEQ ID NO. 109.

(SEQ ID NO. 109)

X₄X₉X₃X₃X₁₀X₇X₄X₁₀
or
X₁₂X₄X₉X₃X₃X₁₀X₇X₄X₁₀
or
X₁₂X₄X₉X₁₁X₁₀X₇X₄X₁₀

An example of the Myelin Proteolipid Protein (MPLP) is the SEQ ID NO. 41 as previously shown.

(SEQ ID NO. 41)

VHFFKNIVTPRTP

The improved variants of Myelin Proteolipid Protein (MPLP) are as follows.

(SEQ ID NO. 114)

X₃X₄X₆X₆X₄X₉X₃X₃X₁₀X₇X₄X₁₀X₇
or
X₁₂X₃X₄X₆X₆X₄X₉X₃X₃X₁₀X₇X₄X₁₀X₇
or
X₁₂X₃X₄X₆X₆X₄X₉X₁₁X₁₀X₇X₄X₁₀X₇

The MBP (Myelin Basic Protein) modified at positions 87-99 is shown by SEQ ID NO. 526 (Hammer et al. (1997), HLA class II peptide binding specificity and autoimmunity, Adv Immunol. 66:67-100).

(SEQ ID NO. 526)

VVHFFKNIVTPR

The improved versions of the MBP (Myelin Basic Protein) modified at positions 87-99 are shown by the following.

(SEQ ID NO. 527)

X₇X₃X₃X₄X₇X₇X₄X₉X₃X₃X₁₀X₇X₄
or

(SEQ ID NO. 528)

X₁₂X₇X₃X₃X₄X₇X₇X₄X₉X₃X₃X₁₀X₇X₄
or

(SEQ ID NO. 529)

X₇X₁₁X₄X₇X₇X₄X₉X₃X₃X₁₀X₇X₄
or

(SEQ ID NO. 530)

X₁₂X₇X₁₁X₄X₇X₇X₄X₉X₃X₃X₁₀X₇X₄
or

(SEQ ID NO. 531)

X₇X₃X₃X₄X₇X₇X₄X₉X₁₁X₁₀X₇X₄
or

(SEQ ID NO. 532)

X₁₂X₇X₃X₃X₄X₇X₇X₄X₉X₁₁X₁₀X₇X₄
or

(SEQ ID NO. 533)

X₇X₃X₃X₄X₇X₇X₄X₉X₃X₃X₁₀X₇X₄
or

(SEQ ID NO. 534)

X₁₂X₇X₃X₃X₄X₇X₇X₄X₉X₃X₃X₁₀X₇X₄
or

(SEQ ID NO. 535)

X₇X₁₁X₄X₇X₇X₄X₉X₁₁X₁₀X₇X₄
or

(SEQ ID NO. 536)

X₁₂X₇X₁₁X₄X₇X₇X₄X₉X₁₁X₁₀X₇X₄

The myelin proteolipid protein PLP₁₃₉₋₁₅₁ (Ruiz et al. (1999) Suppressive immunization with DNA encoding a self-peptide prevents autoimmune disease: modulation of T cell costimulation. J Immunol. 162:3336-41).

(SEQ ID NO. 537)

HSLGKWLGHPDKF

The improved versions of the myelin proteolipid protein PLP₁₃₉₋₁₅₁ are shown by the following.

(SEQ ID NO. 538)

X₄X₁₀X₃X₁X₄X₆X₃X₁X₄X₇X₂X₄X₇
or

(SEQ ID NO. 539)

X₁₂X₄X₁₀X₃X₁X₄X₆X₃X₁X₄X₇X₂X₄X₇

The epitope for Experimental Allergic neuritis from peripheral nerve P2 60-70 or EAN 60-70 is shown by SEQ ID NO. 541 (Araga et al., (1999) A complementary peptide vaccine that induces T cell anergy and prevents experimental allergic neuritis in Lewis rats. J Immunol. 163(1):476-82).

What is claimed is:

1. An immunomodulatory peptide construct having the formula
P₁-x-P₂
where
P₁ is a peptide associated with autoimmune disease, allergy, asthma, host-versus-graft rejection, myocarditis, diabetes, and immune-mediated disease, which binds to an antigen receptor on a set or subset of T cells;
P₂ is a peptide which will cause a Tₕ2 directed immune response by said set or subset of T cells to which the peptide P₁ is attached or which will bind to a T cell receptor which will cause said set or subset of T cells to which the peptide P₁ is attached to initiate, but not complete, an immune response causing said set or subset of T cells to undergo anergy and apoptosis; and
x is a direct bond or linker for covalently bonding P₁ and P₂.

2. The immunomodulatory peptide construct of claim 1 having the formula
P₁-x-P₂
where
P₁ is a peptide associated with autoimmune disease, allergy, asthma, host-versus-graft rejection, myocarditis, diabetes, and immune-mediated disease, which binds to an antigen receptor on a set or subset of T cells selected from the group consisting of SEQ ID NO. 53, SEQ ID NO. 54, SEQ ID NO. 55, SEQ ID NO. 56, SEQ ID NO. 57, SEQ ID NO. 58, SEQ ID NO. 59, SEQ ID NO. 60, SEQ ID NO. 61, SEQ ID NO. 62, SEQ ID NO. 63, SEQ ID NO. 64, SEQ ID NO. 65, SEQ ID NO. 66, SEQ ID NO. 67, SEQ ID NO. 68, SEQ ID NO. 69, SEQ ID NO. 70, SEQ ID NO. 71, SEQ ID NO. 72, SEQ ID NO. 73, SEQ ID NO. 74, SEQ ID NO. 75, SEQ ID NO. 76, SEQ ID NO. 77, SEQ ID NO. 78, SEQ ID NO. 79, SEQ ID NO. 80, SEQ ID NO. 81, SEQ ID NO. 82, SEQ ID NO. 83, SEQ ID NO. 84, SEQ ID NO. 85, SEQ ID NO. 86, SEQ ID NO. 87, SEQ ID NO. 88, SEQ ID NO. 89, SEQ ID NO. 90, SEQ ID NO. 91, SEQ ID NO. 92, SEQ ID NO. 93, SEQ ID NO. 94, SEQ ID NO. 95, SEQ ID NO. 96, SEQ ID NO. 97, SEQ ID NO. 98, SEQ ID NO. 99, SEQ ID NO. 100, SEQ ID NO. 101, SEQ ID NO. 102, SEQ ID NO. 103, SEQ ID NO. 104, SEQ ID NO. 105, SEQ ID NO. 106, SEQ ID NO. 107, SEQ ID NO. 108, SEQ ID NO. 509, SEQ ID NO. 525, SEQ ID NO. 2, SEQ ID NO. 109, SEQ ID NO. 41, SEQ ID NO. 110, SEQ ID NO. 112, SEQ ID NO. 113, SEQ ID NO. 20, SEQ ID NO. 41, SEQ ID NO. 114, SEQ ID NO. 526, SEQ ID NO. 527, SEQ ID NO. 528, SEQ ID NO. 529, SEQ ID NO. 530, SEQ ID NO. 531, SEQ ID NO. 532, SEQ ID NO. 533, SEQ ID NO. 534, SEQ ID NO. 535, SEQ ID NO. 536, SEQ ID NO. 537, SEQ ID NO. 538, SEQ ID NO. 539, SEQ ID NO. 42, SEQ ID NO. 115, SEQ ID NO. 44, SEQ ID NO. 116, SEQ ID NO. 45, SEQ ID NO. 117, SEQ ID NO. 46, SEQ ID NO. 118, SEQ ID NO. 47, SEQ ID NO. 119 and SEQ ID NO. 49.

3. The immunomodulatory peptide construct of claim 1 having the formula
P₁-x-P₂
where
P₂ is a peptide which will cause a Tₕ2 directed immune response by said set or subset of T cells to which the peptide P₁ is attached or which binds to a T cell receptor causing said set or subset of T cells to which the peptide P₁ is attached to initiate, but not complete, an immune response causing said set or subset of T cells to undergo anergy and apoptosis selected from the group consisting of SEQ ID NO. 15, SEQ ID NO. 50, SEQ ID NO. 4, SEQ ID NO. 51, SEQ ID NO. 463, SEQ ID NO. 5, SEQ ID NO. 18, SEQ ID NO. 129, SEQ ID NO. 21, SEQ ID NO. 13 and SEQ ID NO. 51.

4. The immunomodulatory peptide construct of claim 1 having the formula
P₁-x-P₂
where P₁ is a peptide associated with Alzheimer's disease, and the peptide construct P₁-x-P₂ is selected from the group consisting of SEQ ID NO. 141, SEQ ID NO. 143, SEQ ID NO. 145, SEQ ID NO. 147, SEQ ID NO. 149, SEQ ID NO. 151, SEQ ID NO. 153, SEQ ID NO. 155, SEQ ID NO. 221, SEQ ID NO. 223, SEQ ID NO. 225, SEQ ID NO. 227, SEQ ID NO. 229, SEQ ID NO. 231, SEQ ID NO. 233, SEQ ID NO. 235, SEQ ID NO. 301, SEQ ID NO. 303, SEQ ID NO. 305, SEQ ID NO. 307, SEQ ID NO. 309, SEQ ID NO. 311, SEQ ID NO. 313, SEQ ID NO. 315, SEQ ID NO. 381, SEQ ID NO. 383, SEQ ID NO. 385, SEQ ID NO. 387, SEQ ID NO. 389, SEQ ID NO. 391, SEQ ID NO. 393, SEQ ID NO. 395 and SEQ ID NO. 800.

5. The immunomodulatory peptide construct of claim 1 having the formula
P₁-x-P₂
where P₁ is a peptide associated with Dilated Cardiac Myopathy or Experimental Autoimmune Myocarditis and the peptide construct P₁-x-P₂ is selected from the group consisting of SEQ ID NO. 157, SEQ ID NO. 159, SEQ ID NO. 161, SEQ ID NO. 163, SEQ ID NO. 237, SEQ ID NO. 239, SEQ ID NO. 241, SEQ ID NO. 243, SEQ ID NO. 317, SEQ ID NO. 319, SEQ ID NO. 321, SEQ ID NO. 323, SEQ ID NO. 397, SEQ ID NO. 399, SEQ ID NO. 401, SEQ ID NO. 403, SEQ ID NO. 818 and SEQ ID NO. 820.

6. The immunomodulatory peptide construct of claim 1 having the formula
P₁-x-P₂
where P₁ is a peptide associated with diabetes and the peptide construct P₁-x-P₂ is selected from the group consisting of SEQ ID NO. 165, SEQ ID NO. 167, SEQ ID NO. 169, SEQ ID NO. 171, SEQ ID NO. 173, SEQ ID NO. 175, SEQ ID NO. 177, SEQ ID NO. 179, SEQ ID NO. 181, SEQ ID NO. 183, SEQ ID NO. 185, SEQ ID NO. 187, SEQ ID NO. 189, SEQ ID NO. 191, SEQ ID NO. 193, SEQ ID NO. 195, SEQ ID NO. 245, SEQ ID NO. 247, SEQ ID NO. 249, SEQ ID NO. 251, SEQ ID NO. 253, SEQ ID NO. 255, SEQ ID NO. 257, SEQ ID NO. 259, SEQ ID NO. 261, SEQ ID NO. 263, SEQ ID NO. 265, SEQ ID NO. 267, SEQ ID NO. 269, SEQ ID NO. 271, SEQ ID NO. 273, SEQ ID NO. 275, SEQ ID NO. 325, SEQ ID NO. 327, SEQ ID NO. 329, SEQ ID NO. 331, SEQ ID NO. 333, SEQ ID NO. 335, SEQ ID NO. 337, SEQ ID NO. 339, SEQ ID NO. 341, SEQ ID NO. 343, SEQ ID NO. 345, SEQ ID NO. 347, SEQ ID NO. 349, SEQ ID NO. 351, SEQ ID NO. 353, SEQ ID NO. 355, SEQ ID NO. 405, SEQ ID NO. 407, SEQ ID NO. 409, SEQ ID NO. 411, SEQ ID NO. 413, SEQ ID NO. 415, SEQ ID NO. 417, SEQ ID NO. 419, SEQ ID NO. 421, SEQ ID NO. 423, SEQ ID NO. 425, SEQ ID NO. 427, SEQ ID NO. 429, SEQ ID NO. 431, SEQ ID NO. 433, SEQ ID NO. 435, SEQ ID NO. 802, SEQ ID NO. 824 and SEQ ID NO. 826.

7. The immunomodulatory peptide construct of claim 1 having the formula
P₁-x-P₂
where P₁ is a peptide associated with Rheumatoid Arthritis and the peptide construct P₁-x-P₂ is selected from the group consisting of SEQ ID NO. 197, SEQ ID NO. 277, SEQ ID NO. 357, SEQ ID NO. 437 and SEQ ID NO. 804.

8. The immunomodulatory peptide construct of claim 1 having the formula
P₁-x-P₂
where P₁ is a peptide associated with Pemphigus Vulgaris and the peptide construct P₁-x-P₂ is selected from the group consisting of SEQ ID NO. 199, SEQ ID NO. 279, SEQ ID NO. 359, SEQ ID NO. 439, SEQ ID NO. 806, SEQ ID NO. 808, SEQ ID NO. 812 and SEQ ID NO. 814.

9. The immunomodulatory peptide construct of claim 1 having the formula
P₁-x-P₂
where P₁ is a peptide associated with Multiple Sclerosis and the peptide construct P₁-x-P₂ is selected from the group consisting of SEQ ID NO. 201, SEQ ID NO. 205, SEQ ID NO. 207, SEQ ID NO. 281, SEQ ID NO. 285, SEQ ID NO. 287, SEQ ID NO. 361, SEQ ID NO. 365, SEQ ID NO. 367, SEQ ID NO. 441, SEQ ID NO. 445 and SEQ ID NO. 447.

10. The immunomodulatory peptide construct of claim 1 having the formula
P₁-x-P₂
where P₁ is a peptide associated with allergic conditions and the peptide construct P₁-x-P₂ is selected from the group consisting of SEQ ID NO. 209, SEQ ID NO. 211, SEQ ID NO. 213, SEQ ID NO. 215, SEQ ID NO. 217, SEQ ID NO. 219, SEQ ID NO. 289, SEQ ID NO. 291, SEQ ID NO. 293, SEQ ID NO. 295, SEQ ID NO. 297, SEQ ID NO. 299, SEQ ID NO. 369, SEQ ID NO. 371, SEQ ID NO. 373, SEQ ID NO. 375, SEQ ID NO. 377, SEQ ID NO. 379, SEQ ID NO. 449, SEQ ID NO. 451, SEQ ID NO. 453, SEQ ID NO. 455, SEQ ID NO. 457 and SEQ ID NO. 459.

11. The immunomodulatory peptide construct of claim 1 having the formula
P₁-x-P₂
where P₁ is a peptide associated with antiphospholipid conditions and the peptide construct P₁-x-P₂ is selected from the group consisting of SEQ ID NO. 203, SEQ ID NO. 283, SEQ ID NO. 363 and SEQ ID NO. 443.

12. The immunomodulatory peptide construct of claim 1 having the formula
P₁-x-P₂
where P₁ is a peptide associated with uveoretinitis and the peptide construct P₁-x-P₂ is selected from the group consisting of SEQ ID NO. 810, SEQ ID NO. 816 and SEQ ID NO. 822.

13. The immunomodulatory peptide construct of claim 1 having the formula
P₁-x-P₂
where P₁ is a peptide associated with psoriasis and the peptide construct P₁-x-P₂ is selected from the group consisting of SEQ ID NO. 828, SEQ ID NO. 830, SEQ ID NO. 831, SEQ ID NO. 832, SEQ ID NO. 834 and SEQ ID NO. 835.

14. An immunomodulatory peptide construct having the formula
P₃-x-P₄
where P₃ is a peptide construct comprised of X₁ to X₁₄ said peptide P₃ being associated with autoimmune disease, allergy, asthma, host-versus-graft rejection, myocarditis, diabetes, and immune-mediated disease, which binds to an antigen receptor on a set or subset of T cells, and
P₄ is a peptide construct comprised of X₁ to X₁₄ causing a Tₕ2 directed immune response by said set or subset of T cells to which the peptide P₃ is attached or which binds to a T cell receptor causing said set or subset of T cells to which the peptide P₃ is attached to initiate, but not complete, an immune response causing said set or subset of T cells to undergo anergy and apoptosis;
X₁ to X₁₀ and X₁₄ describe a group of amino acids based on their features and X₁₁ to X₁₃ describe modifications to the peptide construct, wherein
X₁ is selected from the group consisting of Ala and Gly,
X₂ is selected from the group consisting of Asp and Glu,
X₃ is selected from the group consisting of Ile, Leu and Val,
X₄ is selected from the group consisting of Lys, Arg and His,
X₅ is selected from the group consisting of Cys and Ser,
X₆ is selected from the group consisting of Phe, Trp and Tyr,
X₇ is selected from the group consisting of Phe and Pro,
X₈ is selected from the group consisting of Met and Nle,
X₉ is selected from the group consisting of Asn and Gln,
X₁₀ is selected from the group consisting of Thr and Ser,
X₁₁ Gaba^{χ} where X₂X₃, X₃X₂, X₂X₃, X₃X₂, X₃X₃, or X₂X₂ can be substituted with X₁₁;
X₁₂ is selected from the group consisting of acetyl, propionyl group, D glycine, D alanine and cyclohexylalanine;
X₁₃ is 5-aminopentanoic where any combination of 3 to 4 amino acids of X₂ and X₃ can be replaced with X₁₃;
X₁₄ is selected from the group consisting of X₁, X₂, X₃, X₄, X₅, X₆, X₇, X₈, X₉ and X₁₀; and
x is a direct bond or linker for covalently bonding P₃ and P₄.

15. The immunomodulatory peptide construct of claim 14 having the formula
P₃-x-P₄
where P₃ is a peptide associated with Alzheimer's disease, and the peptide construct P₃-x-P₄ is selected from the group consisting of SEQ ID NO. 142, SEQ ID NO. 144, SEQ ID NO. 146, SEQ ID NO. 148, SEQ ID NO. 150, SEQ ID NO. 152, SEQ ID NO. 154, SEQ ID NO. 156, SEQ ID NO. 222, SEQ ID NO. 224, SEQ ID NO. 226, SEQ ID NO. 228, SEQ ID NO. 230, SEQ ID NO. 232, SEQ ID NO. 234, SEQ ID NO. 236, SEQ ID NO. 302, SEQ ID NO. 304, SEQ ID NO. 306, SEQ ID NO. 308, SEQ ID NO. 310, SEQ ID NO. 312, SEQ ID NO. 314, SEQ ID NO. 316, SEQ ID NO. 382, SEQ ID NO. 384, SEQ ID NO. 386, SEQ ID NO. 388, SEQ ID NO. 390, SEQ ID NO. 392, SEQ ID NO. 394 and SEQ ID NO. 396.

16. The immunomodulatory peptide construct of claim 14 having the formula
P₃-x-P₄
where P₃ is a peptide associated with Dilated Cardiac Myopathy or Experimental Autoimmune Myocarditis and the peptide construct P₃-x-P₄ is selected from the group consisting of SEQ ID NO. 158, SEQ ID NO. 160, SEQ ID NO. 162, SEQ ID NO. 164, SEQ ID NO. 238, SEQ ID NO. 240, SEQ ID NO. 242, SEQ ID NO. 244, SEQ ID NO. 318, SEQ ID NO. 320, SEQ ID NO. 322, SEQ ID NO. 324, SEQ ID NO. 398, SEQ ID NO. 400, SEQ ID NO. 402, SEQ ID NO. 404, SEQ ID NO. 801, SEQ ID NO. 819 and SEQ ID NO. 821.

17. The immunomodulatory peptide construct of claim 14 having the formula
P₃-x-P₄
where P₃ is a peptide associated with diabetes and the peptide construct P₃-x-P₄ is selected from the group consisting of SEQ ID NO. 166, SEQ ID NO. 168, SEQ ID NO. 170, SEQ ID NO. 172, SEQ ID NO. 174, SEQ ID NO. 176, SEQ ID NO. 178, SEQ ID NO. 180, SEQ ID NO. 182, SEQ ID NO. 184, SEQ ID NO. 186, SEQ ID NO. 188, SEQ ID NO. 190, SEQ ID NO. 192, SEQ ID NO. 194, SEQ ID NO. 196, SEQ ID NO. 246, SEQ ID NO. 248, SEQ ID NO. 250, SEQ ID NO. 252, SEQ ID NO. 254, SEQ ID NO. 256, SEQ ID NO. 258, SEQ ID NO. 260, SEQ ID NO. 262, SEQ ID NO. 264, SEQ ID NO. 266, SEQ ID NO. 268, SEQ ID NO. 270, SEQ ID NO. 272, SEQ ID NO. 274, SEQ ID NO. 276, SEQ ID NO. 326, SEQ ID NO. 328, SEQ ID NO. 330, SEQ ID NO. 332, SEQ ID NO. 334, SEQ ID NO. 336, SEQ ID NO. 338, SEQ ID NO. 340, SEQ ID NO. 342, SEQ ID NO. 344, SEQ ID NO. 346, SEQ ID NO. 348, SEQ ID NO. 350, SEQ ID NO. 352, SEQ ID NO. 354, SEQ ID NO. 356, SEQ ID NO. 406, SEQ ID NO. 408, SEQ ID NO. 410, SEQ ID NO. 412, SEQ ID NO. 414, SEQ ID NO. 416, SEQ ID NO. 418, SEQ ID NO. 420, SEQ ID NO. 422, SEQ ID NO. 424, SEQ ID NO. 426, SEQ ID NO. 428, SEQ ID NO. 430, SEQ ID NO. 432, SEQ ID NO. 434, SEQ ID NO. 436, SEQ ID NO. 803, SEQ ID NO. 825 and SEQ ID NO. 827.

18. The immunomodulatory peptide construct of claim 14 having the formula
P₃-x-P₄
where P₃ is a peptide associated with Rheumatoid Arthritis and the peptide construct P₃-x-P₄ is selected from the group consisting of SEQ ID NO. 198, SEQ ID NO. 278, SEQ ID NO. 358, SEQ ID NO. 438 and SEQ ID NO. 805.

19. The immunomodulatory peptide construct of claim 14 having the formula
P₃-x-P₄
where P₃ is a peptide associated with Pemphigus Vulgaris and the peptide construct P₃-x-P₄ is selected from the group consisting of SEQ ID NO. 200, SEQ ID NO. 280, SEQ ID NO. 360, SEQ ID NO. 440, SEQ ID NO. 807, SEQ ID NO. 809, SEQ ID NO. 813 and SEQ ID NO. 815.

20. The immunomodulatory peptide construct of claim 14 having the formula
P₃-x-P₄
where P₃ is a peptide associated with Multiple Sclerosis and the peptide construct P₃-x-P₄ is selected from the group consisting of SEQ ID NO. 202, SEQ ID NO. 206, SEQ ID NO. 208, SEQ ID NO. 282, SEQ ID NO. 286, SEQ ID NO. 288, SEQ ID NO. 362, SEQ ID NO. 366, SEQ ID NO. 368, SEQ ID NO. 442, SEQ ID NO. 446 and SEQ ID NO. 448.

21. The immunomodulatory peptide construct of claim 14 having the formula
P₃-x-P₄
where P₃ is a peptide associated with allergic conditions and the peptide construct P₃-x-P₄ is selected from the group consisting of SEQ ID NO. 210, SEQ ID NO. 212, SEQ ID NO. 214, SEQ ID NO. 216, SEQ ID NO. 218, SEQ ID NO. 220, SEQ ID NO. 290, SEQ ID NO. 292, SEQ ID NO. 294, SEQ ID NO. 296, SEQ ID NO. 298, SEQ ID NO. 300, SEQ ID NO. 370, SEQ ID NO. 372, SEQ ID NO. 374, SEQ ID NO. 376, SEQ ID NO. 378, SEQ ID NO. 380, SEQ ID NO. 450, SEQ ID NO. 452, SEQ ID NO. 454, SEQ ID NO. 456, SEQ ID NO. 458 and SEQ ID NO. 460.

22. The immunomodulatory peptide construct of claim 14 having the formula
P₃-x-P₄
where P₃ is a peptide associated with antiphospholipid conditions and the peptide construct P₃-x-P₄ is selected from the group consisting of SEQ ID NO. 204, SEQ ID NO. 284, SEQ ID NO. 364, and SEQ ID NO. 444.

23. The immunomodulatory peptide construct of claim 14 having the formula
P₃-x-P₄
where P₃ is a peptide associated with uveoretinitis and the peptide construct P₃-x-P₄ is selected from the group consisting of SEQ ID NO. 811, SEQ ID NO. 817 and SEQ ID NO. 823.

24. The immunomodulatory peptide construct of claim 14 having the formula
P₃-x-P₄
where P₃ is a peptide associated with psoriasis and the peptide construct P₃-x-P₄ is selected from the group consisting of SEQ ID NO. 829 and SEQ ID NO. 833.
